(19) United States
(12) Patent Application Publication
(10) Pub. No.: US 2021/0106281 A1
(43) Pub. Date: Apr. 15, 2021

(54) IMPLANTABLE MEDICAL SYSTEM

(71) Applicant: Bao Q. Tran, Saratoga, CA (US)

(72) Inventor: Bao Q. Tran, Saratoga, CA (US)

(21) Appl. No.: 16/600,491

(22) Filed: Oct. 12, 2019

(51) Int. Cl. A61B 5/00; A61B 5/0205; A61B 5/07; A61B 5/0404; A61B 5/113

(52) U.S. Cl. CPC A61B 5/6861; A61B 5/02055; A61B 5/076; A61B 5/686; A61B 5/6805; A61B 5/02438; A61B 5/113; A61B 5/4839; A61B 5/7267; A61B 5/7282; A61B 5/486; A61B 5/0404

(57) ABSTRACT

A system to monitor a biological subject includes an implantable device to be inserted inside the subject, the device including an implanted transceiver, an accelerometer, one or more sensors, a battery to power the transceiver, accelerometer and one or more sensors, and a wireless charger to charge the battery; and a wireless charging system outside of the subject to charge the battery in the implantable device. Drug(s) may be carried in reservoir(s) and dispensed based on sensor output.

This invention relates generally to systems and methods for remote monitoring, and more particularly to an implantable physiological monitoring and/or medication system.

### BRIEF SUMMARY

In one aspect, a system to monitor a biological subject includes an implantable device to be inserted inside the subject, the device including an implanted transceiver, an accelerometer, one or more sensors, a battery to power the transceiver, accelerometer and one or more sensors, and a wireless charger to charge the battery; and a wireless charging system outside of the subject to charge the battery in the implantable device. Drug(s) may be carried in reservoir(s) and dispensed based on sensor output.

In another aspect, a system to monitor a subject includes sensing a glucose level; if the glucose level is above a predetermined limit, requesting the subject to exercise or perform one or more activities; detecting physical activity or exercise; and repeating the steps until the glucose level is below the predetermined limit.

In yet another aspect, a system to treat a subject includes sensing a glucose level; if the glucose level is above a predetermined limit, requesting the subject to exercise or perform one or more activities; injecting insulin into the subject; detecting physical activity or exercise; and repeating until the glucose level is below the predetermined limit. The system may be an implantable device, or skin mounted.

In a further aspect, a system to monitor an animal includes an implantable device to be inserted inside the animal, the device including an implanted transceiver, an accelerometer, one or more sensors, a battery to power the transceiver, accelerometer and one or more sensors, and a wireless charger to charge the battery; and a wireless charging system outside of the animal to charge the battery in the implantable device.

In yet another aspect, a system to monitor an animal includes an implantable device to be inserted inside the animal, the device including an inductive transceiver to transfer power and data, an accelerometer, one or more sensors, a battery coupled to the inductive transceiver, accelerometer and one or more sensors, and where the inductive transceiver exchanges data with an external unit using a predetermined protocol while the coil is charging the battery. The external unit has a battery and long range transceiver such as a satellite modem that wirelessly communicates with the implantable device while charging the implantable device.

In a further aspect, embodiments of one embodiment provide a system with one or more injectable detecting systems having a plurality of sensors that provide an indication of at least one physiological event of an animal, a wireless communication device coupled to the one or more injectable detecting systems and configured to transfer animal data directly or indirectly from the one or more injectable detecting systems to a remote monitoring system, and a remote monitoring system coupled to the wireless communication device, the remote monitoring system using processed data to determine animal health status for timely treatment to save the animal.

Implementations of the above aspect may include one or more of the following. A glucose sensor communicates data to a remote device to coordinate physical activity or exercise proximal to a meal to adjust glucose level without medication. A Generative Adversarial Networks (GANs), a recurrent neural network, a statistical recognizer, a learning machine, or a neural network can determine health issue from the sensor. One or more medical reservoirs and one or more pumps can dispense medication. The medication can include insulin, blood pressure medication, stroke medication, coronary artery medication, cancer medication, respiratory medication, obstructive pulmonary medication, and Alzheimer medication. A glucose sensor coupled to an insulin reservoir can dispense insulin in a closed loop. A pacemaker can be connected to the glucose sensor, wherein pacemaker operation is adjusted based on glucose level.

Other implementations can include the following. The device is implanted proximal to a shoulder blade or a dorsal midline of the animal. The device is implanted proximal to a neck area, a shoulder blade area, or an area of the animal not accessible to the animal through chewing or biting. The device can include a temperature sensor, heart rate sensor, a hydration sensor, impedance sensor, EKG sensor, or a pulse oximetry sensor. The device alternatively can have a temperature sensor, heart rate sensor, and a pulse oximetry sensor. The device can include a blood pressure sensor or a glucose sensor. A pulse oximetry sensor can be sensed by processor and such output can be used to determine breathing rate from the pulse oximetry sensor. The pulse oximetry sensor can have sensors behind a windowed portion to detect blood flow through the arteries of the animal and such blood flow information is used to determine oxygen level and/or breathing rate. The implanted transceiver can be a personal area network such as Bluetooth or a wireless local area network such as WiFi or can be connected to wired Ethernet if needed. The wireless charger can be an inductive charger or a capacitive charger. A pacemaker circuit can be provided in the device to provide pace making electrical pulses if needed. A neck strap or a vest can be worn by the animal to charge the battery via a strap area within charging range of the wireless charger. The wireless charging system is carried by the vest. A cellular transceiver or a satellite network transceiver can be positioned in the vest to provide global transmission of data by the accelerometer or sensor. The vest comprises a temperature sensor and an EKG sensor. A positioning system can be mounted in the vest/neck strap. Power saving circuit can shut down sensors when not needed to reduce power consumption. The implantable device can be charged by a cellular device during data transmission. The sensor data can be processed by a processor, a statistical recognizer, a learning machine, or a neural network to determine health issue from the accelerometer or sensor. A recurrent neural network can process health data. For highly mobile situations, the implanted transceiver can be a cellular transceiver or a mesh network transceiver to increase range.

In many embodiments, the one or more injectable detecting systems are inserted below the skin of the animal by at least one of, catheter delivery, blunt tunneling and needle insertion.

In many embodiments, the systems and methods further comprise an imaging system to assist in guiding the injectable detecting system to a desired location.

In many embodiments, each of a sensor is selected from at least one of, bioimpedance, heart rate, heart rhythm, HRV, HRT, heart sounds, respiratory sounds, respiratory rate and respiratory rate variability, blood pressure, activity, posture, wake/sleep, orthopnea, temperature, heat flux and an accelerometer.

In many embodiments, each of a sensor is an activity sensors selected from at least one of, ball switch, accelerometer, minute ventilation, HR, bioimpedance noise, skin temperature/heat flux, blood pressure, muscle noise and posture.

In many embodiments, the injectable detecting systems include a power source, a memory, logic resources and an antenna. In many embodiments the power source is a rechargeable battery transcutaneously with an external unit. In many embodiments, at least a portion of the injectable detecting systems have a drug eluting coating.

In many embodiments, the one or more injectable detecting systems are anchored in the animal by at least one of, barbs, anchors, tissue adhesion pads, suture loops, shape of device, self-expanding metal structure, wherein self-expanding metal structure is made of Nitinol.

Advantages of the system may include one or more of the following. The system provides timely monitoring of the animal and protects the animal from physical failures that may lead to traumatic injury, impaired athletic performance, or environmental injuries. The system allows multiple users to access and review the data for each animal at the same time on a secure system. Users can access and review the data from various locations. Data can be analyzed and alerts immediately sent when abnormalities are detected. The system can effectively operate in harsh environmental conditions. The system provides real-time monitoring of the animal's heart rate, body temperature, respiratory rate. Data can be sent from various environments including buildings, outdoors, etc. The implant is durable and lasts for a year or more without requiring replacement. Frequent monitoring of animals permits the animals' physician to detect worsening symptoms as they begin to occur, rather than waiting until a critical condition has been reached. As such, monitoring of animals is becoming increasingly popular in the health care industry for the array of benefits it has the potential to provide. Potential benefits of home monitoring are numerous and include: better tracking and management of disease conditions, earlier detection of changes in the animal condition, and reduction of overall health care expenses associated with long term disease management.

### BRIEF DESCRIPTION OF THE DRAWINGS

FIG. 1A illustrates one embodiment of an animal monitoring system.

FIG. 1B illustrates another embodiment of an animal monitoring system.

FIG. 1C illustrates yet another embodiment of an animal monitoring system.

FIG. 1D illustrates one embodiment of an animal wireless recharging mat.

FIGS. 1E-1F illustrate various neck strap embodiments for animal monitoring system.

FIG. 1G shows exemplary implantation sites for the monitoring device.

FIG. 1H shows another embodiment of an animal monitoring system.

FIG. 1I-1J show an exemplary implanted artificial pancreas.

FIG. 2A shows in more detail an implantable device.

FIG. 2B shows a learning system for recommending treatment based on sensor data captured over time and based on treatment data for a population of animals.

FIG. 3A shows exemplary deep learning systems for recommending treatment from sensor data while FIGS. 3B-3J show alternative exemplary deep learning systems for treatment recommendation.

### DETAILED DESCRIPTION

The one embodiment is directed to a remote animal monitoring/management system that continuously monitors physiological parameters, communicates wirelessly with a remote center, and provides alerts when necessary. A variety of delivery devices and methods are also disclosed.

One embodiment provides a remote physiologic monitoring capability to enhance animal care and capabilities through continuous health monitoring. The ability to provide continuous physiologic monitoring of an MPC at rest, as well as during high levels of performance in all environmental conditions will significantly improve their operational effectiveness, recovery, and overall care. The system provides ability to remotely monitor the physiologic status of animals utilizing an implantable device for collection and transmission of data in real-time, under all environmental conditions. The Implants do not cause tissue reactivity or other bodily harm to the animals.

The system monitors physiological parameters and uses a proprietary algorithm to determine animal health. The injectable system communicates with a remote center, preferably via an intermediate device in the animal's home. In some embodiments, the remote center receives the data and applies a learning machine prediction algorithm. When a flag is raised, the center may communicate with the animal, hospital, nurse, and/or physician to allow for therapeutic intervention to prevent problems.

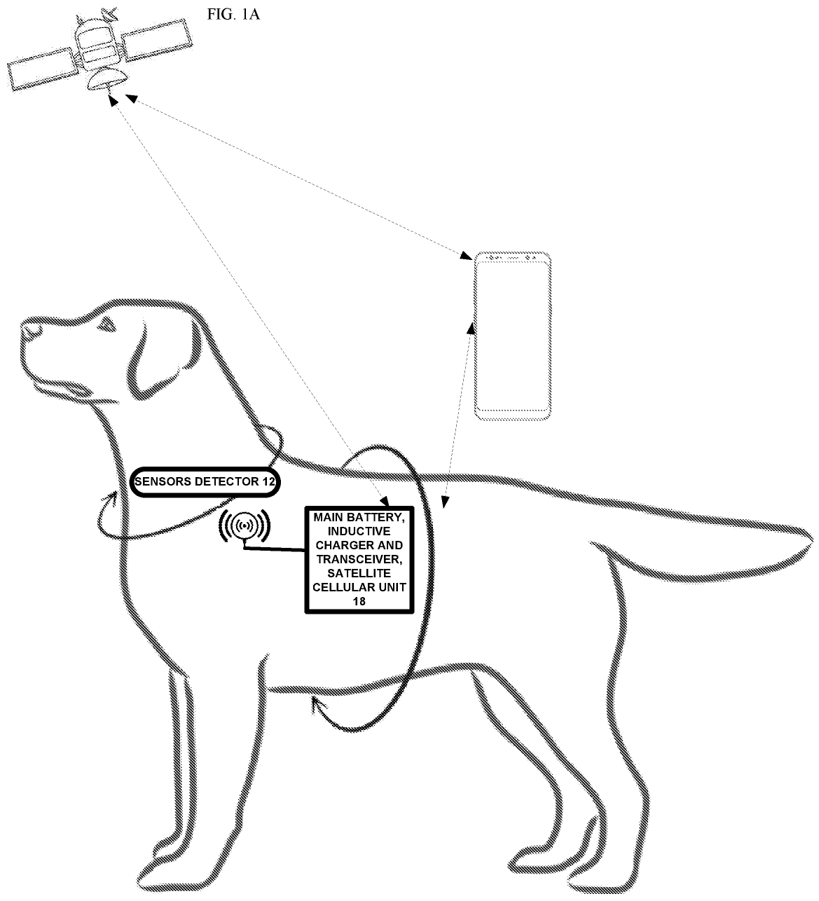

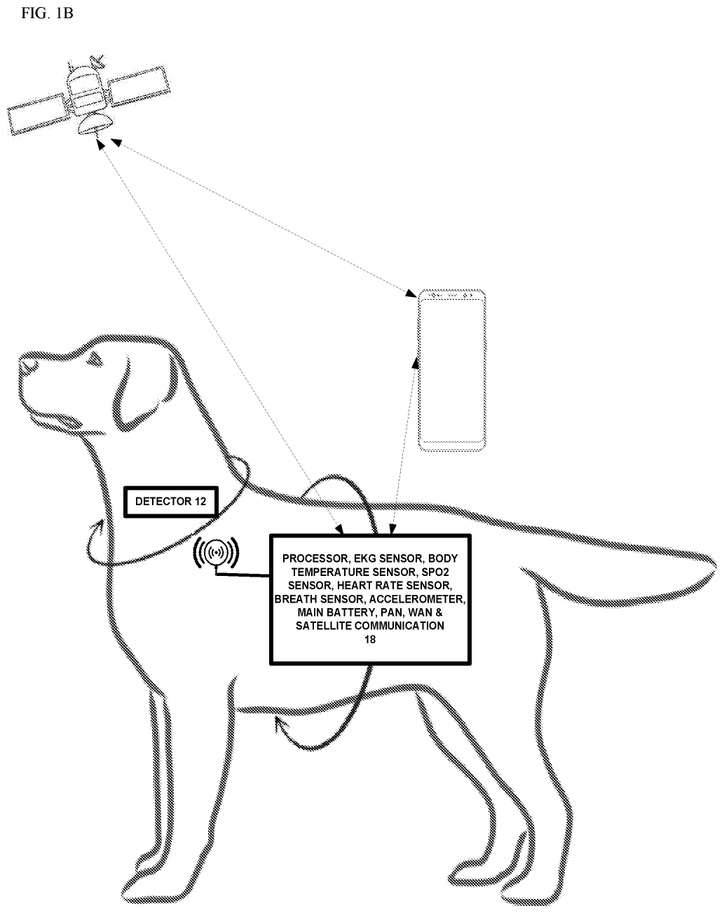

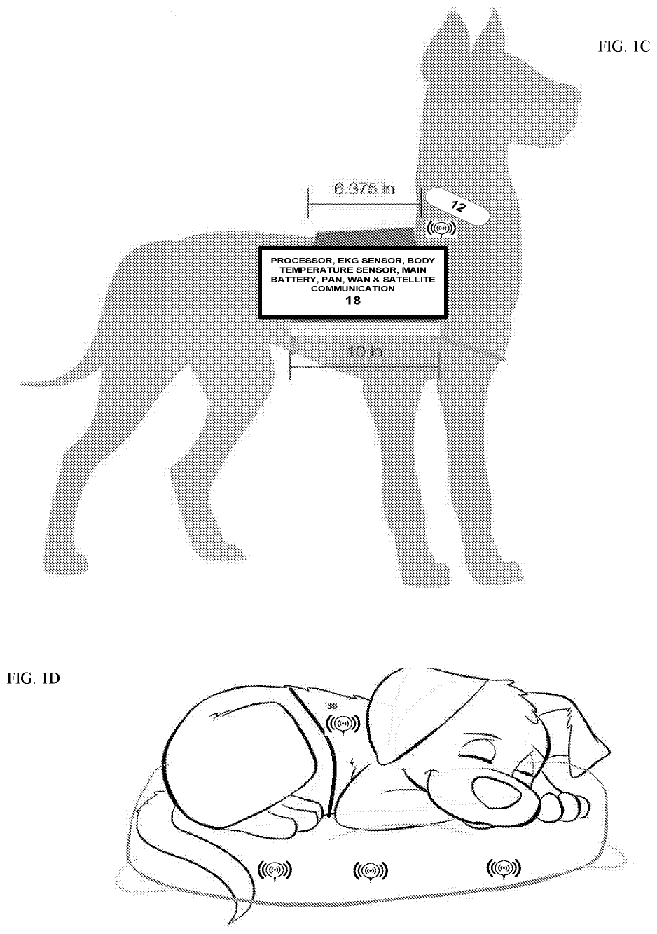

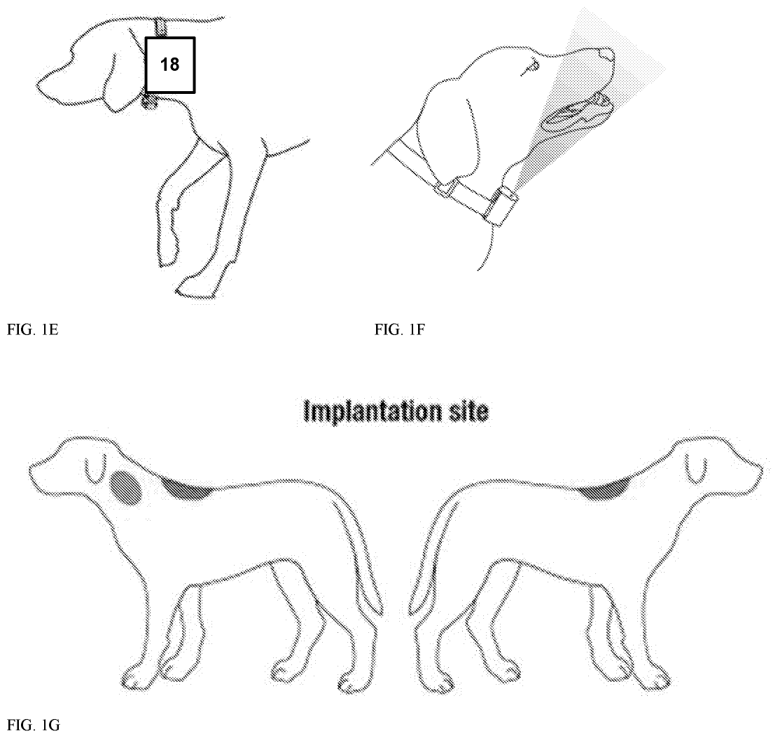

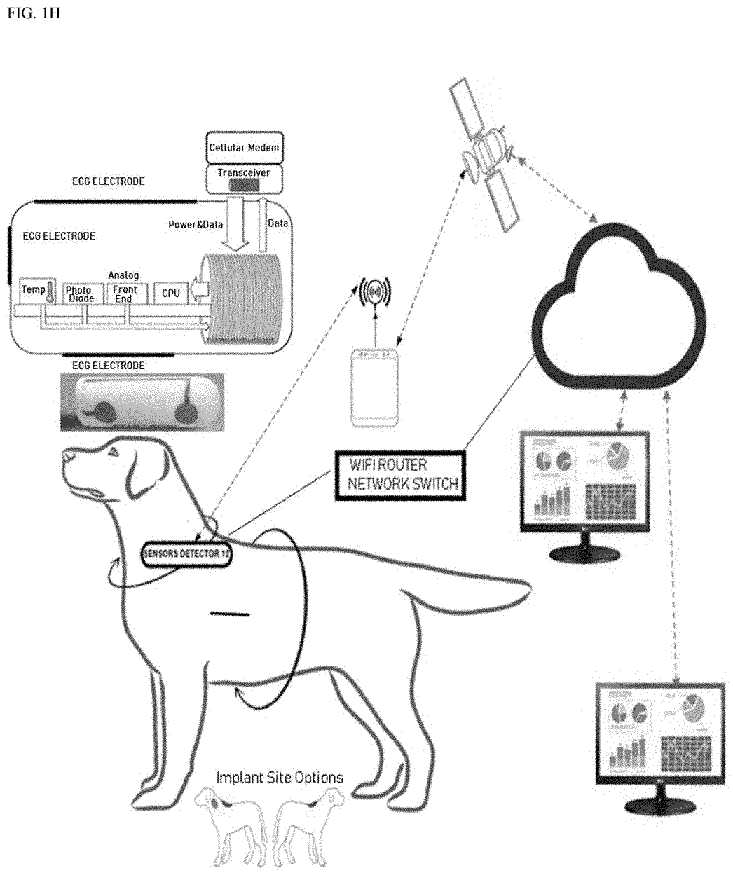

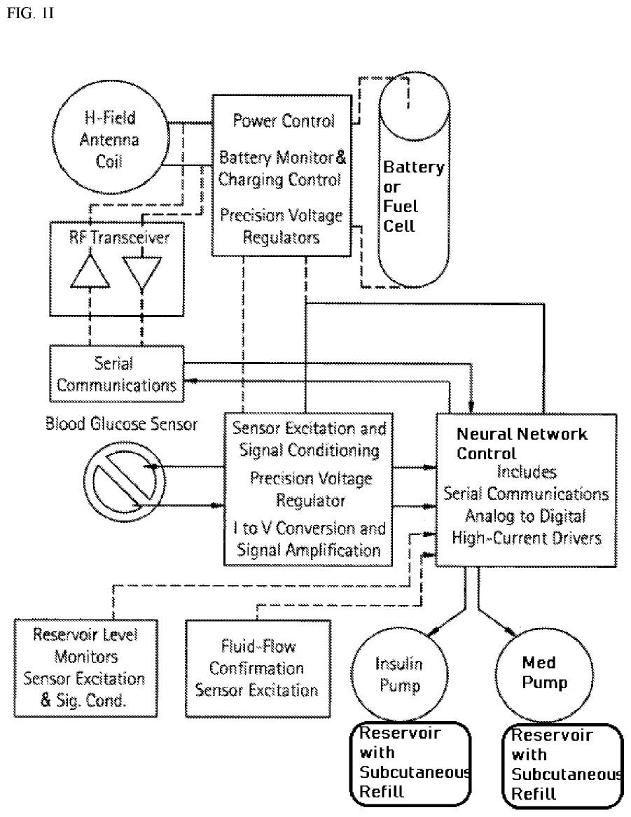

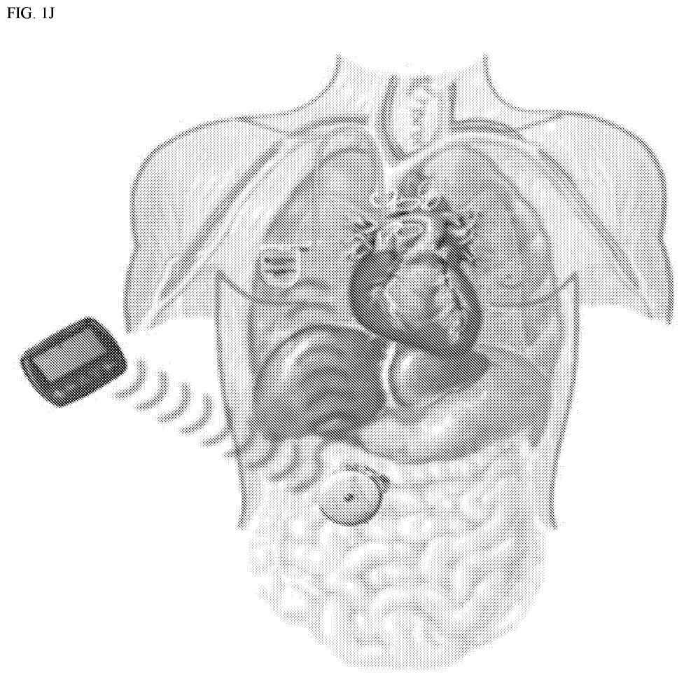

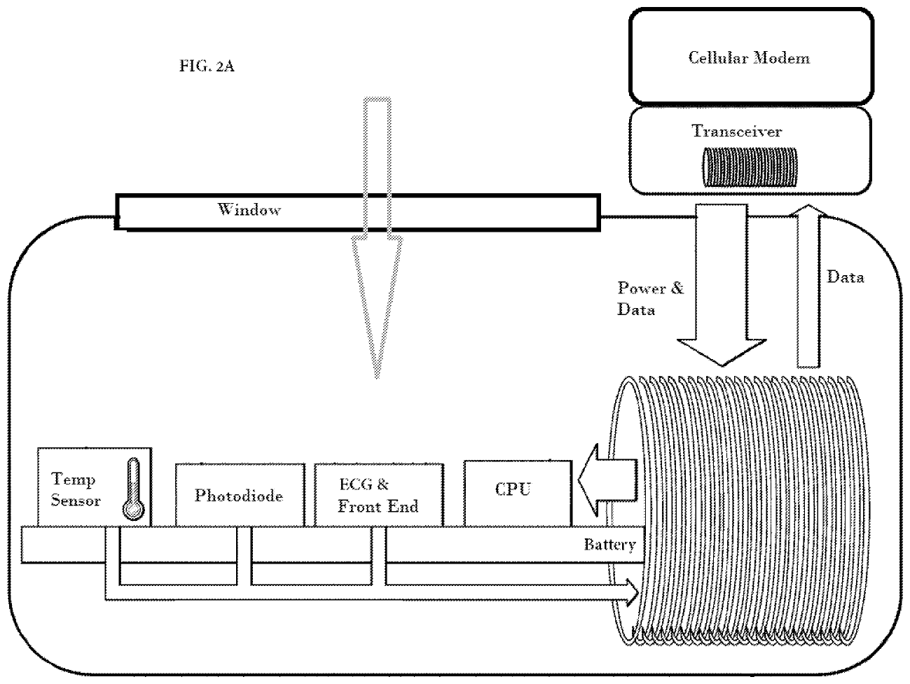

FIG. 1A shows one embodiment where the implantable device 12 contains all sensors, and rely on external unit 18 for power and long-range communication. The system to monitor an animal includes an implantable device 12 to be inserted inside the animal, the device including an inductive transceiver to transfer power and data, an accelerometer, one or more sensors, a battery coupled to the inductive transceiver, accelerometer and one or more sensors, and where the inductive transceiver exchanges data with an external unit using a predetermined protocol while the coil is charging the battery. The external unit 18 has a battery and long-range transceiver such as a satellite modem that wirelessly communicates with the implantable device while charging the implantable device. In contrast, FIG. 1H shows another embodiment of an animal monitoring system where the reader/interrogator is connected to a smart phone or a router, and the implant directly communicates with the smart phone with a minimum radius of one meter to read the data as data must be read when the dog and reader are in close proximity or when the dog is working. In one embodiment, the implant measures ECG from electrodes on its housing in FIG. 1H, and the ECG measurement is transmitted via Bluetooth or WiFi to a Bluetooth or Wifi router that communicates with cloud server via the internet. In one implementation, the implant measures ECG and communicates over Bluetooth to a collar with a Bluetooth to Wifi adapter or Wifi gateway bluetooth/bridge BLE beacons receiver. The Wifi adapter or gateway in turn communicates over WiFi to a router connected to the internet to communicate data with cloud based storage/processor such as Amazon cloud. In one embodiment, the Wifi adapter is a bluetooth 5.0 low energy 5.0 low energy (BLE) to Wi-Fi connectivity gateway without the uses of smartphones or apps. The G1 Gateway collects the data from ibeacon, Eddystone, BLE sensor and other BLE devices, and then sends to the local TCP server or remote cloud server by HTTP/MQTT/mbed (ARM) protocol over Wi-Fi/Ethernet Cellular. The user can configure the gateway via a web UI.

In another embodiment, illustrated in FIGS. 1B-1C, is a system that delivers a percutaneous sensing device for remote animal monitoring where the external unit also has its own sensors such as EKG, body temperature, among others. The distribution of the sensors between implanted device 12 and the external device 18 can be done for battery optimization and circuit advantages. For example, the external unit can have 3 lead EKG circuit that the implanted device cannot easily do. Moreover, breathing pattern can be detected by detecting accelerometer variations in the diaphragm as the animal breathes in and out. Further, the external device 18 can have multiple temperature sensing points, for example. The remote monitoring tracks the animal's physiological status, detects and predicts negative physiological events. In one embodiment, the implanted sensing device includes a plurality of sensors that are used in combination to enhance detection and prediction capabilities as more fully explained below.

In some embodiments, the device 12 has a subcutaneously implantable sensor that measures analyte (e.g., glucose) concentrations in a medium (e.g., interstitial fluid) of a living animal. However, this is not required, and, in some alternative embodiments, the device 12 may be a partially implantable (e.g., transcutaneous) sensor or a fully external sensor.

In some embodiments, the transceiver may be an externally worn transceiver (e.g., attached via an armband, wristband, waistband, or adhesive patch). In some embodiments, the transceiver may remotely power and/or communicate with the sensor to initiate and receive the measurements (e.g., via near field communication (NFC)). However, this is not required, and, in some alternative embodiments, the transceiver may power and/or communicate with the sensor via one or more wired connections. In some non-limiting embodiments, the transceiver may be a smartphone (e.g., an NFC-enabled smartphone). In some embodiments, the transceiver may communicate information (e.g., one or more analyte concentrations) wirelessly (e.g., via a Bluetooth™ communication standard such as, for example and without limitation Bluetooth Low Energy) to a primary display device (e.g., smartphone, tablet, laptop, personal computer, iPod, or health monitoring watch).

In some embodiments, the transceiver may include an inductive element, such as, for example, a coil. The transceiver may generate an electromagnetic wave or electrodynamic field (e.g., by using a coil) to induce a current in an inductive element of the sensor, which powers the sensor. The transceiver 20 may also convey data (e.g., commands) to the sensor. For example, in a non-limiting embodiment, the transceiver may convey data by modulating the electromagnetic wave used to power the sensor (e.g., by modulating the current flowing through a coil of the transceiver). The modulation in the electromagnetic wave generated by the transceiver may be detected/extracted by the sensor. Moreover, the transceiver may receive sensor data (e.g., measurement information) from the 110. For example, in a non-limiting embodiment, the transceiver may receive sensor data by detecting modulations in the electromagnetic wave generated by the sensor, e.g., by detecting modulations in the current flowing through the coil of the transceiver.

The inductive element of the transceiver and the inductive element of the sensor may be in any configuration that permits adequate field strength to be achieved when the two inductive elements are brought within adequate physical proximity.

The sensor can be a temperature sensor, heart rate sensor, a hydration sensor, impedance sensor, EKG sensor, or a pulse oximetry sensor. Other embodiments of device can have a temperature sensor, heart rate sensor, and a pulse oximetry sensor. The device can include a blood pressure sensor using a flow sensor embedded in the vein. The device can also include a glucose sensor. A pulse oximetry sensor can provide Sp02 data that can be processed by processor and such output can additionally be used to determine breathing rate from the pulse oximetry sensor. The pulse oximetry sensor can have sensors behind a windowed portion to detect blood flow through the arteries of the animal and such blood flow information is used to determine oxygen level and/or breathing rate. The implanted transceiver can be a personal area network such as Bluetooth or a wireless local area network such as wifi or can be connected to wired Ethernet if needed. The injectable detecting system 12 can include one or more rechargeable batteries that can be transcutaneously chargeable with an external unit. The wireless charger can be an inductive charger or a capacitive charger.

The device includes power and communication elements, and a communication antenna. The antenna may be a self-expanding antenna expandable from a first compressed shape to a second expanded shape. An energy management device can control power to the plurality of sensors. In one embodiment, the energy management device is part of the detecting system. In various embodiments, the energy management device performs one or more of, modulate drive levels per sensed signal of a sensor, modulate a clock speed to optimize energy, watch cell voltage drop—unload cell, coulomb-meter or other battery monitor, sensor dropoff at an end of life of a battery coupled to a sensor, battery end of life dropoff to transfer data, elective replacement indicator, call center notification, sensing windows by the sensors based on a monitored physiological parameter and sensing rate control. Rechargeable battery is used, but in various embodiments, the energy management device is configured to manage energy by at least one of, a thermo-electric unit, kinetics, fuel cell, nuclear power, a micro-battery and with a rechargeable device.

Referring again to FIGS. 1B-1C, in one embodiment, the system 10 includes an injectable detecting system 12 that includes a plurality of sensors and/or electrodes, that provide an indication of at least one physiological event of an animal. The injectable detecting system 12 is inserted subcutaneously. In one embodiment the injectable detecting system 12 is inserted in the animal's shoulder blade, dorsal midline, or the thorax, among others. For canine, the dorsal midline is preferred. The system 10 also includes a wireless communication device 16, coupled to the plurality of sensors 14. The wireless communication device transfers animal data directly or indirectly from the plurality of sensors to a remote monitoring system 18. The remote monitoring system 18 uses data from the sensors to determine the animal's status. The system 10 can continuously, or non-continuously, monitor the animal, alerts are provided as necessary and medical intervention is provided when required. In one embodiment, the wireless communication device is a wireless local area network for receiving data from the plurality of sensors. The wireless communication can also be cellular or satellite communication for extended range monitoring.

A neck strap or a vest can be worn by the animal to charge the battery via a strap area within charging range of the wireless charger. The wireless charging system is carried by the vest. A cellular transceiver or a satellite network transceiver can be positioned in the vest to provide global transmission of data by the accelerometer or sensor. The vest comprises a temperature sensor and an EKG sensor. A positioning system can be mounted in the vest/neck strap. Power saving circuit can shut down sensors when not needed to reduce power consumption. The implantable device can be charged by a cellular device during data transmission. The sensor data can be processed by a processor, a statistical recognizer, a learning machine, or a neural network to determine health issue from the accelerometer or sensor. A recurrent neural network can process health data. For highly mobile situations, the implanted transceiver can be a cellular transceiver or a mesh network transceiver to increase range.

A processor is coupled to the plurality of sensors in the injectable detecting system 12. The processor receives data from the plurality of sensors and creates processed animal data. In one embodiment, the processor is at the remote monitoring system 18. In another embodiment, the processor is at the detecting system 12. The processor can be integral with a monitoring unit 22 that is part of the injectable detecting system 12 or part of the remote monitoring system 18.

The processor has program instructions for evaluating values received from the sensors with respect to acceptable physiological ranges for each value received by the processor and determine variances. The processor can receive and store a sensed measured parameter from the sensors 14, compare the sensed measured value with a predetermined target value, determine a variance, accept and store a new predetermined target value and also store a series of questions from the remote monitoring system 18.

The injectable detecting system 12 can provide a variety of different functions, including but not limited to, initiation, programming, measuring, storing, analyzing, communicating, predicting, and displaying of a physiological event of the animal. The injectable detecting system 12 can be sealed, such as housed in a hermetically sealed package. In one embodiment, at least a portion of the sealed packages include a power source, a memory, logic resources and a wireless communication device. In one embodiment, an antenna is included that is exterior to the sealed package of the injectable detecting system 12. In one embodiment, the sensors include, flex circuits, thin film resistors, organic transistors and the like. The sensors can include ceramics, titanium PEEK, along with a silicon, PU or other insulative adherent sealant, to enclose the electronics. Additionally, the injectable detecting system 12 can include drug eluting coatings, including but not limited to, an antibiotic, anti-inflammatory agent and the like.

A wide variety of different sensors can be utilized, including but not limited to, bioimpedance, heart rate, heart rhythm, HRV, HRT, heart sounds, respiration rate, respiration rate variability, respiratory sounds, SpO2, blood pressure, activity, posture, wake/sleep, orthopnea, temperature, heat flux, an accelerometer. glucose sensor, other chemical sensors associated with cardiac conditions, and the like. A variety of activity sensors can be utilized, including but not limited to a, ball switch, accelerometer, minute ventilation, HR, bioimpedance noise, skin temperature/heat flux, BP, muscle noise, posture and the like.

The output of the sensors can have multiple features to enhance physiological sensing performance. These multiple features have multiple sensing vectors that can include redundant vectors. The sensors can include current delivery electrodes and sensing electrodes. Size and shape of current delivery electrodes, and the sensing electrodes, can be optimized to maximize sensing performance. The system 10 can be configured to determine an optimal sensing configuration and electronically reposition at least a portion of a sensing vector of a sensing electrode. The multiple features enhance the system's 10 ability to determine an optimal sensing configuration and electronically reposition sensing vectors. In one embodiment, the sensors can be partially masked to minimize contamination of parameters sensed by the sensors 14.

The size and shape of current delivery electrodes, for bioimpedance, and sensing electrodes can be optimized to maximize sensing performance Additionally, the outputs of the sensors can be used to calculate and monitor blended indices. Examples of the blended indices include but are not limited to, heart rate (HR) or respiratory rate (RR) response to activity, HR/RR response to posture change, HR+RR, HR/RR+bioimpedance, and/or minute ventilation/accelerometer and the like.

The sensors can be cycled in order to manage energy, and different sensors can sample at different times. By way of illustration, and without limitation, instead of each sensor being sampled at a physiologically relevant interval, e.g. every 30 seconds, one sensor can be sampled at each interval, and sampling cycles between available sensors.

By way of illustration, and without limitation, the sensors can sample 5 seconds for every minute for ECG, once a second for an accelerometer sensor, and 10 seconds for every 5 minutes for impedance.

In one embodiment, a first sensor is a core sensor that continuously monitors and detects, and a second sensor verifies a physiological status in response to the core sensor raising a flag. Additionally, some sensors can be used for short term tracking, and other sensors used for long term tracking.

The device is implanted proximal to a shoulder blade or a dorsal midline of the animal. The device is implanted proximal to a neck area, a shoulder blade area, or an area of the animal not accessible to the animal through chewing or biting. The sensors are subcutaneously inserted with the injectable detecting system 12 that is catheter based, blunt tunneling (with either a separate tunneling tool or a wire-stiffened lead), needle insertion gun or syringe-like injection. The injectable detecting system 12 can be flexible, and be used with a stiffening wire, stylet, catheter or guidewire. The injectable detecting system 12 can include any of the following to assist in subsequent extraction: (i) an isodiametric profile, (ii) a breakaway anchor, (iii) a bioabsorbable material, (iv) coatings to limit tissue in-growth, (v) an electrically activated or fusable anchor, and the like. The injectable detecting system 12 can be modular, containing multiple connected components, a subset of which is easily extractable.

The injectable detecting system 12 can be inserted in the animal in a non-sterile or sterile setting, non-surgical setting or surgical setting, implanted with or without anesthesia and implanted with or without imaging assistance from an imaging system. The injectable detecting system 12 can be anchored in the animal by a variety of means including but not limited to, barbs, anchors, tissue adhesion pads, suture loops, with sensor shapes that conform to adjacent tissue anatomy or provide pressure against the adjacent tissue, with the use of self-expanding materials such as a nitinol anchor and the like.

The system can be configured to automatically detect events. The system 12 automatically detects events by at least one of, high noise states, physiological quietness, sensor continuity and compliance. In response to a detected physiological event, animal states are identified when data collection is inappropriate. In response to a detected physiological event, animal states are identified when data collection is desirable. Animal states include, physiological quietness, rest, relaxation, agitation, movement, lack of movement and an animal's higher level of animal activity.

A shown in FIG. 1D, in one embodiment, recharging coils are placed in a mat on the animal's bed, such as under a mattress pad. Recharging of the sensors/battery and data transfer can occur during sleep of the animal. The rechargeable batteries can be transcutaneously charged with an external unit other than the mattress. Data from the sensors can be transferred during sleep of the animal. The injectable detecting system 12 downloads data to the mat and a modem such as 5G modem or satellite phone is used for data transfer. In one embodiment, the wireless communication device is configured to receive instructional data from the remote monitoring system and communicate instructions to the injectable detecting system.

In one embodiment, the injectable detecting system 12 communicates with the remote monitoring system 18 periodically or in response to a trigger event. The trigger event can include but is not limited to at least one of, time of day, if a memory is full, if an action is initiated by the dog owner, if an action is initiated from the remote monitoring system, a diagnostic event of the monitoring system, an alarm trigger, a mechanical trigger, and the like.

The injectable detecting system 12 can continuously, or non-continuously, monitor the animal, alerts are provided as necessary and medical intervention is provided when required. In one embodiment, the wireless communication device is a wireless local area network for receiving data from the plurality of sensors in the injectable detecting system.

The injectable detecting system 12 is inserted into the animal by a variety of means, including but not limited to, catheter delivery, blunt tunneling, insertion with a needle, by injection, with a gun or syringe device with a stiffening wire and stylet and the like. The sensors can be inserted in the animal in a non-sterile or sterile setting, non-surgical setting or surgical setting, injected with our without anesthesia and injected with or without imaging assistance. The injectable detecting system 12 can be anchored in the animal by a variety of means including but not limited to, barbs, anchors, tissue adhesion pads, suture loops.

The injectable detecting system 12 can come in a variety of different form factors including but not limited to, cylinder, dog-bone, half dog-bone, trapezoidal cross-section, semicircular cross-section, star-shaped cross-section, v-shaped cross-section, L-shaped, canted, W shaped, or in other shapes that assist in their percutaneous delivery, S-shaped, sine-wave shaped, J-shaped, any polygonal shape, helical/spiral, fin electrodes, and linear device with a radius of curvature to match a radius of the injection site and the like. Further, the injectable detecting system 12 can have flexible body configurations. Additionally, the injectable detecting system 12 can be configured to deactivate selected sensors to reduce redundancy.

FIGS. 1E and 1F show exemplary neck strap embodiments that are smaller than vest embodiments. In FIG. 1F, the remote unit 18 is carried on the neck strap and communicates with the implanted unit 12 to monitor the animal health conditions.

In FIG. 1F, the neck strap includes a camera that captures ambient information such as the animal's breathing rate and the food the animal eats, for example. Such images are then processed by neural networks to provide additional confirmation data on animal health. For example, the camera can estimate the amount of food and liquid intake by the animal. The camera can monitor drooling. Anything that prevents the animal from swallowing normally can lead to drool, as the saliva will build up until it drips from his mouth. The problem could be a fractured tooth or tumors inside the mouth, esophagus, and/or throat. Tartar buildup and irritation of the gums can also lead to drooling, as can an infection in the mouth. In addition, a foreign body can lead to slobbering. Anything caught between the teeth or lodged in his throat, such as a sliver of bone, could be a potentially serious problem. Anything that upsets the animal's stomach may lead to slobbering. If the animal eats something he shouldn't, like a sock or the stuffing from a toy, that can also lead to stomach distress and drooling. Additionally, toxic substances can cause drooling. For example, if the animal gets into a poisonous plant in the garden or cleaning chemicals under the sink, slobbering along with other symptoms such as vomiting, shaking, or lethargy can be detected by the camera. Heat stroke, for example, can lead to drooling as the animal pants in an attempt to cool off. After suffering a seizure, the animal may drool. Nose, throat, or sinus infections, or a neuromuscular condition (palsy, tetany, botulism, etc.) of some kind can also lead to slobbering. Kidney disease, liver disease, and even rabies all share drooling as a symptom. The camera can also detect abnormal saliva, such as foul smelling saliva, thicker saliva, or blood in the saliva. In case the animal is injured, the images taken by the camera can be helpful in reconstructing the threat, for example.

The device and sensors can be made of a variety of materials, including but not limited to, silicone, polyurethane, Nitinol, a biocompatible material, a bioabsorbable material and the like. Electrode sensors can have a variety of different conductors, including but not limited to, platinum, MP35N which is a nickel-cobalt-chromium-molybdenum alloy, MP35N/Ag core, platinum/tantalum core, stainless steel, titanium and the like. The sensors can have insulative materials, including but not limited to, polyetheretherketone (PEEK), ethylene-tetrafluoroethylene (ETFE), polytetrafluoroethlene (PTFE), polyimide, silicon, polyurethane, and the like. Further, the sensors can have openings, or an absorbent material, configured to sample a hydration level or electrolyte level in a surrounding tissue site at the location of the sensor 14. The sensor electrodes can be made of a variety of materials, including but not limited to platinum, iridium, titanium, and the like. Electrode coatings can be included, such as iridium oxide, platinum black, TiN, and the like.

One embodiment provides pace making currents to the heart. There are certain breeds with a genetic predisposition to heart abnormalities such as sick sinus syndrome, and if the heart stops for over eight seconds, the dog will pass out. Sometimes an electrical impulse from another part of the heart will trigger a beat to prevent complete arrest. Some animals have a consistent, abnormally slow heartbeat (sinus bradycardia) as the result of a low firing rate from the sinus node. Even during exercise or when excited, the dog's heart rate will be under 40 beats per minute. Other dogs with the condition will have episodes of rapid heartbeat (excessive tachycardia), plus long pauses. English Springer Spaniels are predisposed to another type of heart problem called atrial standstill with missing P-waves, which are a measure of electrical activity in the atria or top two chambers of the heart, and may also show a slow heart rate with either regular or irregular rhythm. An advanced AV block (also called a complete or third-degree block) means the electrical impulses transmitted by the SA node are blocked at the AV node, which causes heart rate abnormalities. Cocker Spaniels, Doberman Pinschers and Pugs are predisposed to the condition, which is seen more often in older dogs. To address these issues, a pacemaker circuit can be provided in the device to provide pace making electrical pulses if needed. The pacemaker circuit is a pulse generator that contacts the myocardium, to deliver a depolarizing pulse and to sense intrinsic cardiac activity. Insulation materials separate the conductor cables and the lead tip electrodes. Depending on the relationship between the cables, the leads can be designed as coaxial (a tube within a tube) or coradial (side-by-side coils). The lead fixation to the myocardium may be active (with an electrically active helix at its tip for mechanical stability) or passive (electrically inert tines anchor the lead). Disruption of conductor elements and insulation materials results in either high impedance (fracture) or low impedance due to short-circuiting (insulation breach), respectively. Pacing occurs when a potential difference (voltage) is applied between the 2 electrodes. In bipolar pacing, the potential difference occurs between the lead tip (cathode) and a proximal ring (anode). With unipolar pacing, current is delivered between the lead tip and the pulse generator can. The minimum amount of energy required to depolarize myocardium is called the stimulation threshold. The delivered stimulus is described by 2 characteristics: its amplitude (measured in volts) and its duration (measured in milliseconds). The energy required to pace the myocardium depends on the programmed pulse width and on the voltage delivered between the electrodes. An exponential relationship (strength-duration curve) exists between the stimulation threshold and the pulse amplitude and duration. This is clinically relevant, in that optimizing the pulse width and amplitude can significantly affect current drain and battery longevity. Another clinical use for these parameters includes reprogramming to prevent extracardiac (e.g., phrenic) stimulation by lowering the pacing voltage to minimize the risk of far-field capture and increasing the pulse width to ensure cardiac stimulation. In some non-limiting embodiments, the electronics may be encased in a sensor housing (i.e., body, shell, capsule, or encasement), which may be biocompatible.

To detect breathing rate, photoplethysmography (PPG) detect changes in the volume of blood flowing through blood vessels due to the rhythmic activity of the heart. This volume change is measured by illuminating the capillary bed with a small light source and measuring the amount of light that reflects or passes through the tissue with a photodiode. The PPG sensing circuit has a photodiode, a transimpedance amplifier (TIA), and two sets of cascaded active filters. Each set of filters is specifically tuned for monitoring heart rate, which has a frequency range of 0.7 Hz to 3.5 Hz corresponding to 42 beats per minute (BPM) to 210 BPM, and respiratory rate, which has a frequency range of 0.2 Hz to 0.5 Hz corresponding to 12 breaths per minute (BrthPM) to 30 BrthPM, for example. The TIA converts the current produced by the photodiode to a voltage. This voltage is then sent into each respective set of active filters tuned for heart and respiratory rate monitoring respectively. The outputs of our PPG sensing circuits are sensed by a microcontroller with a 10-bit analog-to-digital converter. The heart rate circuit is sampled at 11.9 Hz, while the respiration circuit is sampled at 4.0 Hz satisfying Nyquist.

In another embodiment, the sensor may be a glucose sensor and may include an analyte indicator element, such as, for example, a polymer graft coated, diffused, adhered, or embedded on or in at least a portion of the exterior surface of the sensor housing. The analyte indicator element (e.g., polymer graft) of the sensor may include indicator molecules (e.g., fluorescent indicator molecules) exhibiting one or more detectable properties (e.g., optical properties) based on the amount or concentration of the analyte in proximity to the analyte indicator element. In some embodiments, the sensor may include a light source that emits excitation light over a range of wavelengths that interact with the indicator molecules. The sensor may also include one or more photodetectors (e.g., photodiodes, phototransistors, photoresistors, or other photosensitive elements). The one or more photodetectors (e.g., photodetector) may be sensitive to emission light (e.g., fluorescent light) emitted by the indicator molecules such that a signal generated by a photodetector (e.g., photodetector) in response thereto that is indicative of the level of emission light of the indicator molecules and, thus, the amount of analyte of interest (e.g., glucose). In some non-limiting embodiments, one or more of the photodetectors (e.g., photodetector) may be sensitive to excitation light that is reflected from the analyte indicator element as reflection light. In some non-limiting embodiments, one or more of the photodetectors may be covered by one or more filters that allow only a certain subset of wavelengths of light to pass through (e.g., a subset of wavelengths corresponding to emission light or a subset of wavelengths corresponding to reflection light) and reflect the remaining wavelengths. In some non-limiting embodiments, the sensor may include a temperature transducer. In some non-limiting embodiments, the sensor may include a drug-eluting polymer matrix that disperses one or more therapeutic agents (e.g., an anti-inflammatory drug). Although in some embodiments, the sensor may be an optical sensor, this is not required, and, in one or more alternative embodiments, sensor may be a different type of analyte sensor, such as, for example, an electrochemical sensor, a diffusion sensor, or a pressure sensor. Also, although in some embodiments, the analyte sensor may be a fully implantable sensor, this is not required, and, in some alternative embodiments, the sensor may be a transcutaneous sensor having a wired connection to the transceiver. For example, in some alternative embodiments, the sensor may be located in or on a transcutaneous needle (e.g., at the tip thereof). In these embodiments, instead of wirelessly communicating using inductive elements, the sensor and transceiver may communicate using one or more wires connected between the transceiver and the transceiver transcutaneous needle that includes the sensor. For another example, in some alternative embodiments, the sensor may be located in a catheter (e.g., for intravenous blood glucose monitoring) and may communicate (wirelessly or using wires) with the transceiver.

In yet another embodiment, the glucose sensor can be combined with a glucose reservoir and pump to be an implantable artificial pancreas. In this embodiment, an implantable insulin pump is surgically implanted under the skin and a catheter from the pump extends into the peritoneal cavity. The insulin delivered to the peritoneal cavity is quickly routed to the liver—the normal destination for insulin. With the implantable insulin pump the liver receives a high concentration of insulin, keeps a large percentage of it and allows only a small amount to pass to the rest of the body. This more closely (than subcutaneous insulin delivery) matches the way insulin is delivered in people who do not have diabetes. Thus, a normal positive portal-peripheral insulin gradient is established. In contrast, subcutaneous (SubQ) insulin delivery does just the opposite and creates an abnormal Negative Portal-Peripheral Insulin Gradient where the insulin concentration reaching the liver is lower than the concentration of insulin in the rest of the body. This contributes to the difficulty of maintaining a stable blood glucose level experienced by Type 1 Diabetes.

In another embodiment shown in FIG. 1I, an artificial pancreas comprises at least one pump, a glucose monitor and the associated electronics, which form a closed loop system that can maintain blood glucose levels at a desired value and additionally reduce the tissue inflammatory response. The device has an inlet port for refilling an insulin reservoir and an inlet for refilling a medication reservoir, such as anti-inflammatory agent reservoir. The refilling port is connected to a subcutaneous injection point via a tube/catheter, and a valve is actuated to direct the refilling to the insulin reservoir or the medication reservoir. The implantable device has a duplex pump to dispense insulin for maintaining blood glucose levels at a desired value and additionally can dispense a therapeutic agent to the site of implantation of a glucose monitor to reduce tissue inflammatory response. The artificial pancreas further comprises an implantable glucose monitor that can advantageously function for an extended period of time when implanted subcutaneously in a living being. The artificial pancreas also comprises suitable electronics that in conjunction with the pump and the glucose monitor form a closed loop system. The artificial pancreas can advantageously be implanted into the body of a living being and can function without maintenance or removal from the body for a time period greater than or equal to about 1 month, preferably greater than or equal to about 6 months, and more preferably greater than or equal to about 12 months. High pressure capabilities of the insulin pump can be utilized to minimize clogging of the lines in the system due to insulin precipitation. If a form of insulin that displays excessive precipitation is used, the artificial pancreas advantageously permits the lines to be periodically flushed with a saline solution injected through a side arm on the capillary by transcutaneous delivery. The artificial pancreas has a long life since the simultaneous delivery of the anti-inflammatory agent to the glucose monitor prevents inflammation at the site at which the monitor is implanted. The artificial pancreas can advantageously be implanted into the body of a living being and can function without maintenance or removal from the body for extended periods of time.

As shown in the schematic in FIG. 1I, the control electronics contained in the electronics bay, provides the interface between the signal generated by the glucose monitor signal and the insulin pump, thereby creating a closed-loop system. A precision voltage source provides the excitation for the monitor, and the battery also provides the current for the heating the film 200 used in the pumps 20A, 20B. The software controls the output rate of both the insulin and the anti-inflammatory agent pumps. The insulin pump may be controlled by a standard proportional-integral-differential (PID) control and adjust the response of the pump so that the delivery of insulin can be adjusted for a time period of about milliseconds to tens of minutes. This response of the pump is adjusted to accommodate different types of insulin that might be used, with the objective of minimizing divergence of glucose levels from the desired 5.5 mmol/l. Similarly the output of the anti-inflammatory agent pump will be selected to match a desired delivery rate in order to minimize inflammation. A neural network is used to learn the medication or insulin injection rate and customize to the patient's body response. The housing of the entry port preferably comprises materials that are biocompatible and through which a hypodermic syringe can be introduced for purposes of replenishing the reservoirs with glucose and the therapeutic agent if desired. Examples of suitable therapeutic agents include anti-inflammatory agents such as dexamethasone, prednisolone, corticosterone, budesonide, estrogen, sulfasalazine, mesalamine, or the like. The preferred anti-inflammatory agent is dexamethasone. The therapeutic agent can be genetic, non-genetic or may comprise cells or cellular matter. Examples of non-genetic therapeutic agents are antithrombogenic agents such as heparin and its derivatives, urokinase, and dextropheylalanine proline arginine chloromethylketone (Ppack); anti-proliferative agents such as enoxaprin, andiopeptin, or monoclonal antibodies capable of blocking smooth muscle cell proliferation, hirudin, and acetylsalicylic acid; antineoplastic/antiproliferative/anti-miotic agents such as paclitaxel, 5-fluorouracil, cisplatin, vinblastine, vincristine, epothilones, endostatin, angiostatine and thymidine kinase inhibitors; anesthetic agents such as lidocaine, bupivacaine, and ropivacaine; anti-coagulants such as D-Phe-Pro-Arg chloromethyl keton, an RGD peptide-containing compound, heparin, antithrombin compounds, platelet receptor antagonists, anti-thrombin anticodies, anti-platelet receptor antibodies, aspirin, prostaglandin inhibitors, platelet inhibitors and tick antiplatelet peptides; vascular cell growth promoters such as growth factor inhibitors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vascoactive mechanisms. In one embodiment, the housing comprises at least one port (not shown) through which additional glucose and anti-inflammatory agent may be added to the first reservoir and the second reservoir respectively for purposes of replenishing the supply. In another embodiment, the housing comprises polymeric resinous materials that are self-curing, wherein the housing upon being impaled by a hypodermic syringe for the purpose of replenishing the glucose or anti-inflammatory agent into the respective reservoirs may undergo self-curing to eliminate the cavity in the housing created by the introduction of the syringe. In yet another embodiment the design can encompass more than one second reservoir for retaining multiple therapeutic agents. In such a case, the additional reservoirs may be designated as a third reservoir, fourth reservoir and so on, depending upon the number of reservoirs. All such reservoirs may be in fluid contact with the pump and can be isolated from one other if desired. If desired, some or all of these additional reservoirs may be in fluid communication with one another through check valves and other associated fluid handling devices such as pumps, gages, valves, nozzles, orifices, and the like. Suitable examples of such metallic biocompatible materials that may be used for the housing and partitions therein are titanium or titanium alloys such as nitinol, stainless steel, tantalum, and cobalt alloys including cobalt-chromium nickel alloys. Suitable nonmetallic biocompatible materials are polymeric resins such as polyamides, polytetrafluoroethylene, silicone polymers such as polydimethylsiloxane, polyolefins such as polyethylene and/or polypropylene, nonabsorbable polyesters such polyethylene terephthalate and/or polybutylene terephthalate and bioabsorbable aliphatic polyesters such as homopolymers and copolymers of lactic acid, glycolic acid, lactide, glycolide, para-dioxanone, trimethylene carbonate, E-caprolactone, or the like, or biocompatible combinations comprising at least one of the foregoing non-metallic biocompatible materials.

FIG. 1J shows a diagrammatic representation of an implantable insulin pump that can work in parallel with a pacemaker. As an insulin shock event can cause cardiovascular failure, the glucose sensor part of the pump can coordinate with the pacemaker/heart implant to regulate heart beats and minimize impacts from glucose level.

In a non-implantable implementation, a bihormonal bionic pancreas in both adults and children uses a removable sensor located in a thin needle inserted under the skin that automatically monitors real time glucose levels in tissue fluid. It also provided insulin and the hormone glucagon, via two automatic pumps.

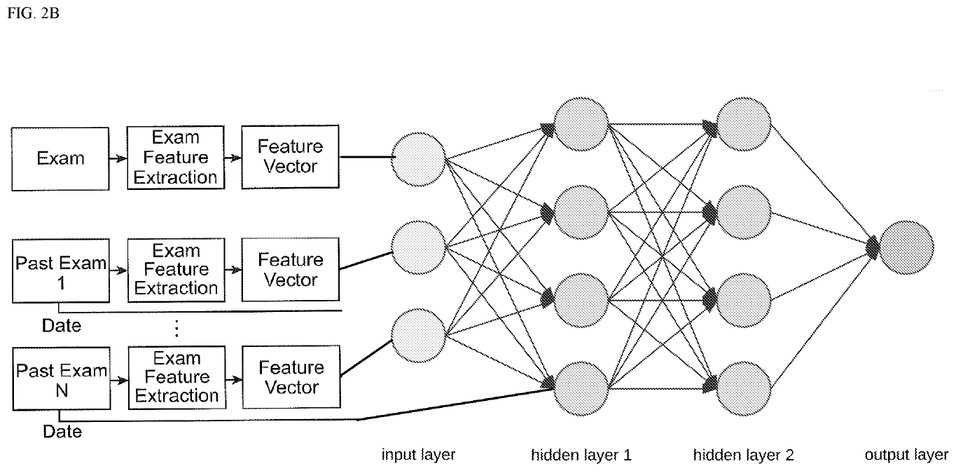

FIG. 2A shows in more detail an implantable device with a combined power and data transceiver. In this system, the housing has a window to detect blood flow using PPG photodiodes. The device has a temperature sensor and photodiodes and an analog front end. The photodiode output is provided to a transimpedance amplifier. The signal from the transimpedance amplifier is sent into two sets of cascaded active filters tuned for heart rate sensing and respiration monitoring respectively. The outputs of the amplifier stages are sensed by two channels of an analog-to-digital converter on a Bluetooth-enabled microcontroller. The signals are processed by the on-board microcontroller. Digitally controlled potentiometers are used enabling automatic gain control. Using these potentiometers, the CPU/microcontroller can modulate system gain in order to adjust for differences in the optical reflective properties of skin across difference subjects. In some embodiments, the sensor may include a transceiver interface device. In some embodiments where the sensor includes an antenna (e.g., inductive element), the transceiver interface device may include the antenna (e.g., inductive element) of sensor. In some of the transcutaneous embodiments where there exists a wired connection between the sensor and the transceiver, the transceiver interface device may include the wired connection.

For initial benchtop validation, the discrete Fourier transform (DFT) can be calculated with 128 samples using a rectangular window for both sets of measurements (heart rate and respiration). The process identifies the local maxima of the resulting spectra and compared those frequencies. The sampling rates and number of samples were chosen in order to optimize frequency resolution as well as the length of sampling period as known to those skilled in the art. To avoid performing compute intensive trigonometric floating-point calculations, the trigonometric relationships are looked up in an array of 128 values mapped as 16-bit unsigned integers between 0 and 1000. Indexing the array instead of computing the exact value of the trigonometric function to increase speed extensively. The calculation of the DFT is limited to the frequencies of interests, namely 0.7 Hz to 3.5 Hz for heart rate measurements and 0.2 Hz to 0.5 Hz for respiratory rate measurements. For increased refresh rate of measurements, the processor can perform sliding DFT computation across the sampling period.

In one embodiment, an injectable device for use in subcutaneous physiological monitoring of an animal, the injectable device includes: a body; a plurality of sensors that provide an indication of at least one physiological event of an animal, wherein the plurality of sensors include current delivery electrodes and sensing electrodes in contact with tissue of the animal and spaced along the body of the injectable device; and a monitoring unit located within the body and coupled to the plurality of sensors and configured to monitor a bioimpedance of the animal using the current delivery electrodes and sensing electrodes, wherein the monitoring unit utilizes the monitored bioimpedance to determine a hydration of surrounding tissue and wherein the monitoring unit is further configured to, based at least in part on the determined hydration, detect an impending cardiac decompensation of the animal.

Implementations can include one or more of the following. An electrocardiogram (EKG) circuitry can measure heart rate, EKG, hydration. The monitoring unit utilizes the measured impedance signal to monitor a respiration signal associated with the animal. The monitoring unit compares the monitored hydration, the respiration signal and the electrocardiogram signal to baseline values established for each, wherein the monitoring unit sets a flag indicating cardiac problem. The accelerometer can be a three-axis accelerometer configured to measure at least one inclination, a position, an orientation, and an acceleration of the animal in three dimensions. The monitoring unit utilizes activity data received from the accelerometer to detect physiological events and identify animal states when data collection is desirable. Animal states in which data collection is desirable include rest states. The injectable device body has a proximal end and a distal end, wherein the distal end is shaped to penetrate tissue. The injectable device can be inserted in the animal in one or more of a non-sterile setting, sterile setting, non-surgical setting, and surgical setting.

In another aspect, an injectable device for use in subcutaneous physiological monitoring of an animal, includes: a hermetically sealed body; a plurality of sensors that provide an indication of at least one physiological event of an animal, wherein the plurality of sensors includes an accelerometer located within the body. The system has two electrodes spaced along the hermetically sealed body of the injectable device and in contact with tissue of the animal for monitoring a physiological signal associated with the animal. The two electrodes are utilized to measure an impedance signal related, wherein the monitoring unit utilizes the measured impedance to determine a hydration of surrounding tissue of the animal and based on the determined hydration and the activity level of the animal, the device can detect an impending cardiac decompensation of the animal. The processor compares the monitored hydration, the respiration signal and the electrocardiogram signal to baseline values established for each, wherein the monitoring unit sets a flag indicating cardiac decompensation based on a combination of the compared values.

An automated reader can be coupled to an auxiliary input in order to allow a single monitoring unit to be used by multiple animals. As previously mentioned above, the monitoring unit can be at the remote monitoring system and each animal can have an animal identifier (ID) including a distinct animal identifier. In addition, the ID identifier can also contain animal specific configuration parameters. The automated reader can scan the animal identifier ID and transmit the animal ID number with an animal data packet such that the main data collection station can identify the animal.

It will be appreciated that other medical treatment devices can also be used. The injectable detecting system 12 can communicate wirelessly with the external devices in a variety of ways including but not limited to, a public or proprietary communication standard and the like. The injectable detecting system 12 can be configured to serve as a communication hub for multiple medical devices, coordinating sensor data and therapy delivery while transmitting and receiving data from the remote monitoring system.

The device can provide notification when values received from the sensors are not within acceptable physiological ranges. A variety of notification devices can be utilized, including but not limited to, a visible animal indicator, an audible alarm, an emergency medical service notification, a call center alert, direct medical provider notification and the like. The notification can be sent to a variety of different entities, including but not limited to, a caregiver, the remote monitoring system, the owner/spouse/family member, a medical provider, from one device to another device such as the external device, and the like.

Notification can be according to a preset hierarchy. By way of illustration, and without limitation, the preset hierarchy can be, animal notification first and medical provider second, animal notification second and medical provider first, and the like. Upon receipt of a notification, a medical provider, the remote monitoring system 18, or a medical treatment device can trigger a high-rate sampling of physiological parameters for alert verification.

The system 10 can also include an alarm for generating a human perceptible signal when values received from the sensors are not within acceptable physiological ranges. The alarm can trigger an event to render medical assistance to the animal, provide notification as set forth above, continue to monitor, wait and see, and the like.

In another embodiment, the injectable detecting system 12 can switch between different modes, wherein the modes are selected from at least one of: a stand alone mode with communication directly with the remote monitoring system 18, communication with an implanted device, communication with a single implanted device, coordination between different devices (external systems) coupled to the plurality of sensors and different device communication protocols.

In one embodiment, the wireless communication device can include a, modem, a controller to control data supplied by the injectable detecting system 12, serial interface, LAN or equivalent network connection and a wireless transmitter. Additionally, the wireless communication device 16 can include a receiver and a transmitter for receiving data indicating the values of the physiological event detected by the plurality of sensors, and for communicating the data to the remote monitoring system 18. Further, the wireless communication device can have data storage for recording the data received from the injectable detecting system 12 and an access device for enabling access to information recording in the data storage from the remote monitoring system 18.

In various embodiments, the remote monitoring system 18 can include a, receiver, a transmitter and a display for displaying data representative of values of the one physiological event detected by the injectable detecting system 12. The remote monitoring system can also include a, data storage mechanism that has acceptable ranges for physiological values stored therein, a comparator for comparing the data received from the injectable detecting system 12 with the acceptable ranges stored in the data storage device and a portable computer. The remote monitoring system 18 can be a portable unit with a display screen and a data entry device for communicating with the wireless communication device 16.

The injectable detecting system 12 can also include a power management module configured to power down certain components of the system, including but not limited to, the analog-to-digital converters, digital memories and the non-volatile data archive memory and the like, between times when these components are in use. This helps to conserve battery power and thereby extend the useful life. Other circuitry and signaling modes may be devised by one skilled in the art.

As previously mentioned, the system 10 uses an intelligent combination of sensors to enhance detection and prediction capabilities. Electrocardiogram circuitry can be coupled to the sensors, or electrodes, to measure an electrocardiogram signal of the animal. An accelerometer can be mechanically coupled, for example adhered or affixed, to the sensors 14, adherent patch and the like, to generate an accelerometer signal in response to at least one of an activity or a position of the animal. The accelerometer signals improve animal diagnosis, and can be especially useful when used with other signals, such as electrocardiogram signals and impedance signals, including but not limited to, hydration respiration, and the like. Mechanically coupling the accelerometer to the sensors 14, electrodes, for measuring impedance, hydration and the like can improve the quality and/or usefulness of the impedance and/or electrocardiogram signals. By way of illustration, and without limitation, mechanical coupling of the accelerometer to the sensors 14, electrodes, and to the skin of the animal can improve the reliability, quality and/or accuracy of the accelerometer measurements, as the sensor 14, electrode, signals can indicate the quality of mechanical coupling of the patch to the animal so as to indicate that the device is connected to the animal and that the accelerometer signals are valid. Other examples of sensor interaction include but are not limited to, (i) orthopnea measurement where the breathing rate is correlated with posture during sleep, and detection of orthopnea, (ii) a blended activity sensor using the respiratory rate to exclude high activity levels caused by vibration (e.g. driving on a bumpy road) rather than exercise or extreme physical activity, (iii) sharing common power, logic and memory for sensors, electrodes, and the like.

The injectable system may be inserted with one of the following techniques: catheter delivery, blunt tunneling (with either a separate tunneling tool or a wire-stiffened lead), and insertion with a needle. The injectable system may be injected with a gun or syringe-like device. The injectable system may be flexible, and may be implanted with a stiffening wire or stylet.

Subcutaneous implantation of sensors can be done with an insertion and tunneling tool. The tunneling tool includes a stylet and a peel-away sheath. The tunneling tool is inserted into an incision and the stylet is withdrawn once the tunneling tool reaches a desired position. An electrode segment is inserted into the subcutaneous tunnel and the peel-away sheath is removed. In another delivery device, a pointed tip is inserted through the skin and a plunger is actuated to drive the sensor to its desired location.

In other delivery systems, an implant trocar includes a cannula for puncturing the skin and an obturator for delivering the implant. A spring element received within the cannula prevents the sensor from falling out during the implant process. Another sensor delivery device includes an injector that has a tubular body divided into two adjacent segments with a hollow interior bore. A pair of laterally adjacent tines extend longitudinally from the first segment to the distal end of the tubular body. A plunger rod has an exterior diameter just slightly larger than the interior diameter of the tubular body. With the second segment inserted beneath the skin, the push rod is advanced longitudinally through the tubular body, thereby pushing the sensor through the bore. As the implant and rod pass through the second segment, the tines are forced radially away from each other, thereby dilating or expanding the incision, and facilitating implant. The instrument is removed from the incision following implantation.

The injectable system may be implanted in a non-sterile, non-surgical setting. Implantation may occur with or without local anesthesia, and with or without imaging assistance.

The system may consist of an implantable component and an external component. In such an embodiment, the injected component, with or without physiological sensing electrodes, would be used to anchor an external electronics unit. The anchoring mechanism may be magnetic or mechanical.

The injectable system may contain one of the following features to facilitate subsequent extraction: an isodiametric profile, a breakaway anchor, a bioabsorbable material, coatings to limit tissue in-growth, and an electrically activated or fusable anchor. The injectable system may be modular, containing multiple connected components, a subset of which is easily extractable.

Exemplary sensors include standard medical diagnostics for detecting the body's electrical signals emanating from muscles (EMG and EOG) and brain (EEG) and cardiovascular system (ECG). Leg sensors can include piezoelectric accelerometers designed to give qualitative assessment of limb movement. Additionally, thoracic and abdominal bands used to measure expansion and contraction of the thorax and abdomen respectively. A small sensor can be mounted on the subject's finger in order to detect blood-oxygen levels and pulse rate. Additionally, a microphone can be attached to throat and used in sleep diagnostic recordings for detecting breathing and other noise. One or more position sensors can be used for detecting orientation of body (lying on left side, right side or back) during sleep diagnostic recordings. Each of sensors can individually transmit data to a server using wired or wireless transmission. Alternatively, all sensors can be fed through a common bus into a single transceiver for wired or wireless transmission. The transmission can be done using a magnetic medium such as a floppy disk or a flash memory card, or can be done using infrared or radio network link, among others. The sensor can also include an indoor positioning system or alternatively a global position system (GPS) receiver that relays the position and ambulatory patterns of the animal or wearer to the server 20 for mobility tracking.

In one embodiment, the back of the device is a conductive metal electrode that in conjunction with a second electrode, enables differential EKG or ECG to be measured. The electrical signal derived from the electrodes is typically 1 mV peak-peak. In one embodiment where only one electrode is available, an amplification of about 1000 is necessary to render this signal usable for heart rate detection. In the embodiment with more than one electrodes, a differential amplifier is used to take advantage of the identical common mode signals from the EKG contact points, the common mode noise is automatically cancelled out using a matched differential amplifier. In one embodiment, the differential amplifier is a Texas Instruments INA321 instrumentation amplifier that has matched and balanced integrated gain resistors. This device is specified to operate with a minimum of 2.7V single rail power supply. The INA321 provides a fixed amplification of 5× for the EKG signal. With its CMRR specification of 94 dB extended up to 3 KHz the INA321 rejects the common mode noise signals including the line frequency and its harmonics. The quiescent current of the INA321 is 40 mA and the shut down mode current is less than 1 mA. The amplified EKG signal is internally fed to the on chip analog to digital converter. The ADC samples the EKG signal with a sampling frequency of 512 Hz. Precise sampling period is achieved by triggering the ADC conversions with a timer that is clocked from a 32.768 kHz low frequency crystal oscillator. The sampled EKG waveform contains some amount of super imposed line frequency content. This line frequency noise is removed by digitally filtering the samples. In one implementation, a 17-tap low pass FIR filter with pass band upper frequency of 6 Hz and stop band lower frequency of 30 Hz is implemented in this application. The filter coefficients are scaled to compensate the filter attenuation and provide additional gain for the EKG signal at the filter output. This adds up to a total amplification factor of greater than 1000× for the EKG signal.

The sensor can be an ultrasound transducer, optical transducer or electromagnetic sensors, among others. In one embodiment, the transducer is an ultrasonic transducer that generates and transmits an acoustic wave upon command from the CPU during one period and listens to the echo returns during a subsequent period. In use, the transmitted bursts of sonic energy are scattered by red blood cells flowing through the subject's radial artery, and a portion of the scattered energy is directed back toward the ultrasonic transducer 84. The time required for the return energy to reach the ultrasonic transducer varies according to the speed of sound in the tissue and according to the depth of the artery. Typical transit times are in the range of 6 to 7 microseconds. The ultrasonic transducer is used to receive the reflected ultrasound energy during the dead times between the successive transmitted bursts. The frequency of the ultrasonic transducer's transmit signal will differ from that of the return signal, because the scattering red blood cells within the radial artery are moving. Thus, the return signal, effectively, is frequency modulated by the blood flow velocity.

A driving and receiving circuit generates electrical pulses which, when applied to the transducer, produce acoustic energy having a frequency on the order of 8 MHz, a pulse width or duration of approximately 8 microseconds, and a pulse repetition interval (PRI) of approximately 16 μs, although other values of frequency, pulse width, and PRI may be used. In one embodiment, the transducer 84 emits an 8 microsecond pulse, which is followed by an 8 microsecond “listen” period, every 16 microseconds. The echoes from these pulses are received by the ultrasonic transducer 84 during the listen period. The ultrasonic transducer can be a ceramic piezoelectric device of the type well known in the art, although other types may be substituted.

An analog signal representative of the Doppler frequency of the echo is received by the transducer and converted to a digital representation by the ADC, and supplied to the CPU for signal processing. Within the CPU, the digitized Doppler frequency is scaled to compute the blood flow velocity within the artery based on the Doppler frequency. Based on the real time the blood flow velocity, the CPU applies the vital model to the corresponding blood flow velocity to produce the estimated blood pressure value.

Prior to operation, calibration is done using a calibration device and the monitoring device to simultaneously collect blood pressure values (systolic, diastolic pressures) and a corresponding blood flow velocity generated by the monitoring device. The calibration device is attached to the base station and measures systolic and diastolic blood pressure using a cuff-based blood pressure monitoring device that includes a motor-controlled pump and data-processing electronics. While the cuff-based blood pressure monitoring device collects animal or wearer data, the transducer collects animal or wearer data in parallel and through the device 12's radio transmitter, blood flow velocity is sent to the base station for generating a computer model that converts the blood flow velocity information into systolic and diastolic blood pressure values and this information is sent wirelessly from the base station to the device 12 for display and to a remote server if needed. This process is repeated at a later time (e.g., 15 minutes later) to collect a second set of calibration parameters. In one embodiment, the computer model fits the blood flow velocity to the systolic/diastolic values. In another embodiment, the computer trains a neural network or HMM to recognize the systolic and diastolic blood pressure values.

After the computer model has been generated, the system is ready for real-time blood pressure monitoring. In an acoustic embodiment, the transducer directs ultrasound at the animal or wearer's artery and subsequently listens to the echoes therefrom. The echoes are used to determine blood flow, which is fed to the computer model to generate the systolic and diastolic pressure values as well as heart rate value. The CPU's output signal is then converted to a form useful to the user such as a digital or analog display, computer data file, or audible indicator. The output signal can drive a speaker to enable an operator to hear a representation of the Doppler signals and thereby to determine when the transducer is located approximately over the radial artery. The output signal can also be wirelessly sent to a base station for subsequent analysis by a physician, nurse, caregiver, or treating professional. The output signal can also be analyzed for medical attention and medical treatment.

It is noted that while the above embodiment utilizes a preselected pulse duration of 8 microseconds and pulse repetition interval of 16 microseconds, other acoustic sampling techniques may be used in conjunction with the invention. For example, in a second embodiment of the ultrasonic driver and receiver circuit (not shown), the acoustic pulses are range-gated with a more complex implementation of the gate logic. As is well known in the signal processing arts, range-gating is a technique by which the pulse-to-pulse interval is varied based on the receipt of range information from earlier emitted and reflected pulses. Using this technique, the system may be “tuned” to receive echoes falling within a specific temporal window which is chosen based on the range of the echo-producing entity in relation to the acoustic source. The delay time before the gate is turned on determines the depth of the sample volume. The amount of time the gate is activated establishes the axial length of the sample volume. Thus, as the acoustic source (in this case the ultrasonic transducer 84) is tuned to the echo-producing entity (red blood cells, or arterial walls), the pulse repetition interval is shortened such that the system may obtain more samples per unit time, thereby increasing its resolution. It will be recognized that other acoustic processing techniques may also be used, all of which are considered to be equivalent.

In the electromagnetic sensor embodiment, the device is a flexible plastic material incorporated with a flexible magnet. The magnet provides a magnetic field, and one or more electrodes similar to electrode are positioned on the device to measure voltage drops which are proportional to the blood velocity. The flexible magnet produces a pseudo-uniform (non-gradient) magnetic field. The magnetic field can be normal to the blood flow direction or may be a rotative pseudo-uniform magnetic field so that the magnetic field is in a transversal direction in respect to the blood flow direction. The electrode output signals are processed to obtain a differential measurement enhancing the signal to noise ratio. The flow information is derived based on the periodicity of the signals. The decoded signal is filtered over several periods and then analyzed for changes used to estimate artery and vein blood flow. Systemic stroke volume and cardiac output may be calculated from the peripheral SV index value.

In one embodiment, Analog Device's AD627, a micro-power instrumentation amplifier, is used for differential recordings while consuming low power. In dual supply mode, the power rails Vs can be as low as ±1.1 Volt, which is ideal for battery-powered applications. With a maximum quiescent current of 85 μA (60 μA typical), the unit can operate continuously for several hundred hours before requiring battery replacement. The batteries are lithium cells providing 3.0V to be capable of recording signals up to +1 mV to provide sufficient margin to deal with various artifacts such as offsets and temperature drifts. The amplifier's reference is connected to the analog ground to avoid additional power consumption and provide a low impedance connection to maintain the high CMRR. To generate virtual ground while providing low impedance at the amplifier's reference, an additional amplifier can be used. In one implementation, the high-pass filtering does not require additional components since it is achieved by the limits of the gain versus frequency characteristics of the instrumentation amplifier. The amplifier has been selected such that with a gain of 60 dB, a flat response could be observed up to a maximum of 100 Hz with gain attenuation above 100 Hz in one implementation. In another implementation, a high pass filter is used so that the cut-off frequency becomes dependent upon the gain value of the unit. The bootstrap AC-coupling maintains a much higher CMRR so critical in differential measurements. Assuming that the skin-electrode impedance may vary between 5 K- and 10 K-ohms, 1 M-ohm input impedance is used to maintain loading errors below acceptable thresholds between 0.5% and 1%.

When an electrode is placed on the skin, the detection surfaces come in contact with the electrolytes in the skin. A chemical reaction takes place which requires some time to stabilize, typically in the order of a few seconds. The chemical reaction should remain stable during the recording session and should not change significantly if the electrical characteristics of the skin change from sweating or humidity changes. The active electrodes do not require any abrasive skin preparation and removal of hair. The electrode geometry can be circular or can be elongated such as bars. The bar configuration intersects more fibers. The inter detection-surface distance affects the bandwidth and amplitude of the EMG signal; a smaller distance shifts the bandwidth to higher frequencies and lowers the amplitude of the signal. An inter detection-surface of 1.0 cm provides one configuration that detects representative electrical activity of the muscle during a contraction. The electrode can be placed between a motor point and the tendon insertion or between two motor points, and along the longitudinal midline of the muscle. The longitudinal axis of the electrode (which passes through both detection surfaces) should be aligned parallel to the length of the muscle fibers. The electrode location is positioned between the motor point (or innervation zone) and the tendinous insertion, with the detection surfaces arranged so that they intersect as many muscle fibers as possible.

In one embodiment, a multi-functional bio-data acquisition provides programmable multiplexing of the same differential amplifiers for extracting EEG (electroencephalogram), ECG (electrocardiogram), or EMG (electromyogram) waves. The system includes an AC-coupled chopped instrumentation amplifier, a spike filtering stage, a constant gain stage, and a continuous-time variable gain stage, whose gain is defined by the ratio of the capacitors. The system consumes microamps from 3V. The gain of the channel can be digitally set to 400, 800, 1600 or 2600. Additionally, the bandwidth of the circuit can be adjusted via the bandwidth select switches for different biopotentials. The high cut-off frequency of the circuit can be digitally selected for different applications of EEG acquisition.

In another embodiment, a high-resolution, rectangular, surface array electrode-amplifier and associated signal conditioning circuitry captures electromyogram (EMG) signals. The embodiment has a rectangular array electrode-amplifier followed by a signal conditioning circuit. The signal conditioning circuit is generic, i.e., capable of receiving inputs from a number of different/interchangeable EMG/EKG/EEG electrode-amplifier sources (including from both monopolar and bipolar electrode configurations). The electrode-amplifier is cascaded with a separate signal conditioner minimizes noise and motion artifact by buffering the EMG signal near the source (the amplifier presents a very high impedance input to the EMG source, and a very low output impedance); minimizes noise by amplifying the EMG signal early in the processing chain (assuming the electrode-amplifier includes signal gain) and minimizes the physical size of this embodiment by only including a first amplification stage near the body. The signals are digitized and transmitted over a wireless network such as WiFi, Zigbee, or Bluetooth transceivers and processed by the base station that is remote from the animal or wearer. For either high-resolution monopolar arrays or classical bipolar surface electrode-amplifiers, the output of the electrode-amplifier is a single-ended signal (referenced to the isolated reference). The electrode-amplifier transduces and buffers the EMG signal, providing high gain without causing saturation due to either offset potentials or motion artifact. The signal conditioning circuit provides selectable gain (to magnify the signal up to the range of the data recording/monitoring instrumentation, high-pass filtering (to attenuate motion artifact and any offset potentials), electrical isolation (to prevent injurious current from entering the subject) and low-pass filtering (for anti-aliasing and to attenuate noise out of the physiologic frequency range).

The EMG signal can be rectified, integrated a specified interval of and subsequently forming a time series of the integrated values. The system can calculate the root-mean-squared (rms) and the average rectified (avr) value of the EMG signal. The system can also determine muscle fatigue through the analysis of the frequency spectrum of the signal. The system can also assess neurological diseases which affect the fiber typing or the fiber cross-sectional area of the muscle. Various mathematical techniques and Artificial Intelligence (AI) analyzer can be applied. Mathematical models include wavelet transform, time-frequency approaches, Fourier transform, Wigner-Ville Distribution (WVD), statistical measures, and higher-order statistics. AI approaches towards signal recognition include Artificial Neural Networks (ANN), dynamic recurrent neural networks (DRNN), fuzzy logic system, Genetic Algorithm (GA), and Hidden Markov Model (HMM).

A single-threshold method or alternatively a double threshold method can be used which compares the EMG signal with one or more fixed thresholds. The embodiment is based on the comparison of the rectified raw signals and one or more amplitude thresholds whose value depends on the mean power of the background noise. Alternatively, the system can perform spectrum matching instead of waveform matching techniques when the interference is induced by low frequency baseline drift or by high frequency noise.

EMG signals are the superposition of activities of multiple motor units. The EMG signal can be decomposed to reveal the mechanisms pertaining to muscle and nerve control. Decomposition of EMG signal can be done by wavelet spectrum matching and principle component analysis of wavelet coefficients where the signal is de-noised and then EMG spikes are detected, classified and separated. In another embodiment, principle components analysis (PAC) for wavelet coefficients is used with the following stages: segmentation, wavelet transform, PCA, and clustering. EMG signal decomposition can also be done using non-linear least mean square (LMS) optimization of higher-order cumulants.

Time and frequency domain approaches can be used. The wavelet transform (WT) is an efficient mathematical tool for local analysis of non-stationary and fast transient signals. One of the main properties of WT is that it can be implemented by means of a discrete time filter bank. The Fourier transforms of the wavelets are referred as WT filters. The WT represents a very suitable method for the classification of EMG signals. The system can also apply Cohen class transformation, Wigner-Ville distribution (WVD), and Choi-Williams distribution or other time-frequency approaches for EMG signal processing.

In Cohen class transformation, the class time-frequency representation is particularly suitable to analyze surface myoelectric signals recorded during dynamic contractions, which can be modeled as realizations of nonstationary stochastic process. The WVD is a time-frequency that can display the frequency as a function of time, thus utilizing all available information contained in the EMG signal. Although the EMG signal can often be considered as quasi-stationary there is still important information that is transited and may be distinguished by WVD. Implementing the WVD with digital computer requires a discrete form. This allows the use of fast Fourier transform (FFT), which produces a discrete-time, discrete-frequency representation. The common type of time frequency distribution is the Short-time Fourier Transform (STFT). The Choi-Williams method is a reduced interference distribution. The STFT can be used to show the compression of the spectrum as the muscle fatigue. The WVD has cross-terms and therefore is not a precise representation of the changing of the frequency components with fatigue. When walls appear in the Choi-William distribution, there is a spike in the original signal. It will decide if the walls contain any significant information for the study of muscle fatigue. In another embodiment, the autoregressive (AR) time series model can be used to study EMG signal. In one embodiment, neural networks can process EMG signal where EMG features are first extracted through Fourier analysis and clustered using fuzzy c-means algorithm. Fuzzy c-means (FCM) is a method of clustering which allows data to belong to two or more clusters. The neural network output represents a degree of desired muscle stimulation over a synergic, but enervated muscle. Error-back propagation method is used as a learning procedure for multilayered, feedforward neural network. In one implementation, the network topology can be the feedforward variety with one input layer containing 256 input neurons, one hidden layer with two neurons and one output neuron. Fuzzy logic systems are advantageous in biomedical signal processing and classification. Biomedical signals such as EMG signals are not always strictly repeatable and may sometimes even be contradictory. The experience of medical experts can be incorporated. It is possible to integrate this incomplete but valuable knowledge into the fuzzy logic system, due to the system's reasoning style, which is similar to that of a animal being. The kernel of a fuzzy system is the fuzzy inference engine. The knowledge of an expert or well-classified examples are expressed as or transferred to a set of “fuzzy production rules” in the form of IF-THEN, leading to algorithms describing what action or selection should be taken based on the currently observed information. In one embodiment, higher-order statistics (HOS) is used for analyzing and interpreting the characteristics and nature of a random process. The subject of HOS is based on the theory of expectation (probability theory).

The device can also include energy harvesters, which can be based on piezoelectric devices, solar cells or electromagnetic devices that convert mechanical vibrations. Power generation with piezoelectrics can be done with body vibrations or by physical compression (impacting the material and using a rapid deceleration using foot action, for example). The vibration energy harvester consists of three main parts. A piezoelectric transducer (PZT) serves as the energy conversion device, a specialized power converter rectifies the resulting voltage, and a capacitor or battery stores the power. The PZT takes the form of an aluminum cantilever with a piezoelectric patch. The vibration-induced strain in the PZT produces an ac voltage. The system repeatedly charges a battery or capacitor, which then operates the EKG/EMG sensors or other sensors at a relatively low duty cycle. In one embodiment, a vest made of piezoelectric materials can be wrapped around a person's chest to generate power when strained through breathing as breathing increases the circumference of the chest for an average animal by about 2.5 to 5 cm. Energy can be constantly harvested because breathing is a constant activity, even when the animal is sedate.

In another embodiment, body energy generation systems include electro active polymers (EAPs) and dielectric elastomers. EAPs are a class of active materials that have a mechanical response to electrical stimulation and produce an electric potential in response to mechanical stimulation. EAPs are divided into two categories, electronic, driven by electric field, and ionic, driven by diffusion of ions. In one embodiment, ionic polymers are used as biological actuators that assist muscles for organs such as the heart and eyes. Since the ionic polymers require a solvent, the hydrated animal body provides a natural environment. Polymers are actuated to contract, assisting the heart to pump, or correcting the shape of the eye to improve vision. Another use is as miniature surgical tools that can be inserted inside the body. EAPs can also be used as artificial smooth muscles, one of the original ideas for EAPs. These muscles could be placed in exoskeletal suits for soldiers or prosthetic devices for disabled persons. Along with the energy generation device, ionic polymers can be the energy storage vessel for harvesting energy. The capacitive characteristics of the EAP allow the polymers to be used in place of a standard capacitor bank.

For wireless nodes that require more power, electromagnetics, including coils, magnets, and a resonant beam, and micro-generators can be used to produce electricity from readily available foot movement. Typically, a transmitter needs about 30 mW, but the device transmits for only tens of milliseconds, and a capacitor in the circuit can be charged using harvested energy and the capacitor energy drives the wireless transmission, which is the heaviest power requirement. Electromagnetic energy harvesting uses a magnetic field to convert mechanical energy to electrical. A coil attached to the oscillating mass traverses through a magnetic field that is established by a stationary magnet. The coil travels through a varying amount of magnetic flux, inducing a voltage according to Faraday's law. The induced voltage is inherently small and must therefore be increased to viably source energy. Methods to increase the induced voltage include using a transformer, increasing the number of turns of the coil, and/or increasing the permanent magnetic field. Electromagnetic devices use the motion of a magnet relative to a wire coil to generate an electric voltage. A permanent magnet is placed inside a wound coil. As the magnet is moved through the coil it causes a changing magnetic flux. This flux is responsible for generating the voltage which collects on the coil terminals. This voltage can then be supplied to an electrical load. Because an electromagnetic device needs a magnet to be sliding through the coil to produce voltage, energy harvesting through vibrations is an ideal application. In one embodiment, electromagnetic devices are placed inside the heel of a shoe. One implementation uses a sliding magnet-coil design, the other, opposing magnets with one fixed and one free to move inside the coil. If the length of the coil is increased, which increases the turns, the device is able to produce more power.

In an electrostatic (capacitive) embodiment, energy harvesting relies on the changing capacitance of vibration-dependant varactors. A varactor, or variable capacitor, is initially charged and, as its plates separate because of vibrations, mechanical energy is transformed into electrical energy. MEMS variable capacitors are fabricated through relatively mature silicon micro-machining techniques.

In another embodiment, the device can be powered from thermal and/or kinetic energy. Temperature differentials between opposite segments of a conducting material result in heat flow and consequently charge flow, since mobile, high-energy carriers diffuse from high to low concentration regions. Thermopiles consisting of n- and p-type materials electrically joined at the high-temperature junction are therefore constructed, allowing heat flow to carry the dominant charge carriers of each material to the low temperature end, establishing in the process a voltage difference across the base electrodes. The generated voltage and power is proportional to the temperature differential and the Seebeck coefficient of the thermoelectric materials. Body heat from the animal is captured by a thermoelectric element whose output is boosted and used to charge the a lithium ion rechargeable battery. The unit utilizes the Seeback Effect which describes the voltage created when a temperature difference exists across two different metals. The thermoelectric generator takes body heat and dissipates it to the ambient air, creating electricity in the process.

In another embodiment, the kinetic energy of animal movement is converted into energy. As the animal move, a small weight inside the device moves like a pendulum and turns a magnet to produce electricity which can be stored in a super-capacitor or a rechargeable lithium battery. Similarly, in a vibration energy embodiment, energy extraction from vibrations is based on the movement of a “spring-mounted” mass relative to its support frame. Mechanical acceleration is produced by vibrations that in turn cause the mass component to move and oscillate (kinetic energy). This relative displacement causes opposing frictional and damping forces to be exerted against the mass, thereby reducing and eventually extinguishing the oscillations. The damping forces literally absorb the kinetic energy of the initial vibration. This energy can be converted into electrical energy via an electric field (electrostatic), magnetic field (electromagnetic), or strain on a piezoelectric material.

Another embodiment extracts energy from the surrounding environment using a small rectenna (microwave-power receivers or ultrasound power receivers) placed in patches or membranes on the skin or alternatively injected underneath the skin. The rectanna converts the received emitted power back to usable low frequency/dc power. A basic rectanna consists of an antenna, a low pass filter, an ac/dc converter and a dc bypass filter. The rectanna can capture renewable electromagnetic energy available in the radio frequency (RF) bands such as AM radio, FM radio, TV, very high frequency (VHF), ultra high frequency (UHF), global system for mobile communications (GSM), digital cellular systems (DCS) and especially the personal communication system (PCS) bands, and unlicensed ISM bands such as 2.4 GHz and 5.8 GHz bands, among others. The system captures the ubiquitous electromagnetic energy (ambient RF noise and signals) opportunistically present in the environment and transforming that energy into useful electrical power. The energy-harvesting antenna is preferably designed to be a wideband, omnidirectional antenna or antenna array that has maximum efficiency at selected bands of frequencies containing the highest energy levels. In a system with an array of antennas, each antenna in the array can be designed to have maximum efficiency at the same or different bands of frequency from one another. The collected RF energy is then converted into usable DC power using a diode-type or other suitable rectifier. This power may be used to drive, for example, an amplifier/filter module connected to a second antenna system that is optimized for a particular frequency and application. One antenna system can act as an energy harvester while the other antenna acts as a signal transmitter/receiver. The antenna circuit elements are formed using standard wafer manufacturing techniques. The antenna output is stepped up and rectified before presented to a trickle charger. The charger can recharge a complete battery by providing a larger potential difference between terminals and more power for charging during a period of time. If battery includes individual micro-battery cells, the trickle charger provides smaller amounts of power to each individual battery cell, with the charging proceeding on a cell by cell basis. Charging of the battery cells continues whenever ambient power is available. As the load depletes cells, depleted cells are switched out with charged cells. The rotation of depleted cells and charged cells continues as required. Energy is banked and managed on a micro-cell basis.

As the energy-harvesting sources supply energy in irregular, random “bursts,” an intermittent charger waits until sufficient energy is accumulated in a specially designed transitional storage such as a capacitor before attempting to transfer it to the storage device, lithium-ion battery, in this case. Moreover, the system must partition its functions into time slices (time-division multiplex), ensuring enough energy is harvested and stored in the battery before engaging in power-sensitive tasks. Energy can be stored using a secondary (rechargeable) battery and/or a supercapacitor. The different characteristics of batteries and supercapacitors make them suitable for different functions of energy storage. Supercapacitors provide the most volumetrically efficient approach to meeting high power pulsed loads. If the energy must be stored for a long time, and released slowly, for example as back up, a battery would be the preferred energy storage device. If the energy must be delivered quickly, as in a pulse for RF communications, but long term storage is not critical, a supercapacitor would be sufficient. The system can employ i) a battery (or several batteries), ii) a supercapacitor (or supercapacitors), or iii) a combination of batteries and supercapacitors appropriate for the application of interest. In one embodiment, a microbattery and a microsupercapacitor can be used to store energy. Like batteries, supercapacitors are electrochemical devices; however, rather than generating a voltage from a chemical reaction, supercapacitors store energy by separating charged species in an electrolyte. In one embodiment, a flexible, thin-film, rechargeable battery from Cymbet Corp. of Elk River, Minn. provides 3.6V and can be recharged by a reader. The battery cells can be from 5 to 25 microns thick. The batteries can be recharged with solar energy, or can be recharged by inductive coupling. The tag is put within range of a coil attached to an energy source. The coil “couples” with the antenna on the RFID tag, enabling the tag to draw energy from the magnetic field created by the two coils.

One embodiment includes bioelectrical impedance (BI) spectroscopy sensors in addition to or as alternates to EKG sensors and heart sound transducer sensors. BI spectroscopy is based on Ohm's Law: current in a circuit is directly proportional to voltage and inversely proportional to resistance in a DC circuit or impedance in an alternating current (AC) circuit. Bioelectric impedance exchanges electrical energy with the animal or wearer body or body segment. The exchanged electrical energy can include alternating current and/or voltage and direct current and/or voltage. The exchanged electrical energy can include alternating currents and/or voltages at one or more frequencies. For example, the alternating currents and/or voltages can be provided at one or more frequencies between 100 Hz and 1 MHz, preferably at one or more frequencies between 5 KHz and 250 KHz. A BI instrument operating at the single frequency of 50 KHz reflects primarily the extra cellular water compartment as a very small current passes through the cell. Because low frequency (<1 KHz) current does not penetrate the cells and that complete penetration occurs only at a very high frequency (>1 MHz), multi-frequency BI or bioelectrical impedance spectroscopy devices can be used to scan a wide range of frequencies.

Bipolar or tetrapolar electrode systems can be used in the BI instruments. Of these, the tetrapolar system provides a uniform current density distribution in the body segment and measures impedance with less electrode interface artifact and impedance errors. In the tetrapolar system, a pair of surface electrodes (I1, I2) is used as current electrodes to introduce a low intensity constant current at high frequency into the body. A pair of electrodes (E1, E2) measures changes accompanying physiological events. Voltage measured across E1-E2 is directly proportional to the segment electrical impedance of the animal subject. Circular flat electrodes as well as band type electrodes can be used. In one embodiment, the electrodes are in direct contact with the skin surface. In other embodiments, the voltage measurements may employ one or more contactless, voltage sensitive electrodes such as inductively or capacitively coupled electrodes. The current application and the voltage measurement electrodes in these embodiments can be the same, adjacent to one another, or at significantly different locations. The electrode(s) can apply current levels from 20 uA to 10 mA rms at a frequency range of 20-100 KHz. A constant current source and high input impedance circuit is used in conjunction with the tetrapolar electrode configuration to avoid the contact pressure effects at the electrode-skin interface.

The BI sensor can be a Series Model which assumes that there is one conductive path and that the body consists of a series of resistors. An electrical current, injected at a single frequency, is used to measure whole body impedance for the purpose of estimating total body water and fat free mass. Alternatively, the BI instrument can be a Parallel BI Model In this model of impedance, the resistors and capacitors are oriented both in series and in parallel in the animal body. Whole body BI can be used to estimate TBW and FFM in healthy subjects or to estimate intracellular water (ICW) and body cell mass (BCM). High-low BI can be used to estimate extracellular water (ECW) and total body water (TBW). Multi-frequency BI can be used to estimate ECW, ICW, and TBW; to monitor changes in the ECW/BCM and ECW/TBW ratios in clinical populations. The instrument can also be a Segmental BI Model and can be used in the evaluation of regional fluid changes and in monitoring extra cellular water in animal or wearers with abnormal fluid distribution, such as those undergoing hemodialysis. Segmental BI can be used to measure fluid distribution or regional fluid accumulation in clinical populations. Upper-body and Lower-body BI can be used to estimate percentage BF in healthy subjects with normal hydration status and fluid distribution. The BI sensor can be used to detect acute dehydration, pulmonary edema (caused by mitral stenosis or left ventricular failure or congestive heart failure, among others), or hyperhydration cause by kidney dialysis, for example. In one embodiment, the system determines the impedance of skin and subcutaneous adipose tissue using tetrapolar and bipolar impedance measurements. In the bipolar arrangement the inner electrodes act both as the electrodes that send the current (outer electrodes in the tetrapolar arrangement) and as receiving electrodes. If the outer two electrodes (electrodes sending current) are superimposed onto the inner electrodes (receiving electrodes) then a bipolar BIA arrangement exists with the same electrodes acting as receiving and sending electrodes. The difference in impedance measurements between the tetrapolar and bipolar arrangement reflects the impedance of skin and subcutaneous fat. The difference between the two impedance measurements represents the combined impedance of skin and subcutaneous tissue at one or more sites. The system determines the resistivities of skin and subcutaneous adipose tissue, and then calculates the skinfold thickness (mainly due to adipose tissue).

Various BI analysis methods can be used in a variety of clinical applications such as to estimate body composition, to determine total body water, to assess compartmentalization of body fluids, to provide cardiac monitoring, measure blood flow, dehydration, blood loss, wound monitoring, ulcer detection and deep vein thrombosis. Other uses for the BI sensor include detecting and/or monitoring hypovolemia, hemorrhage or blood loss. The impedance measurements can be made sequentially over a period of in time; and the system can determine whether the subject is externally or internally bleeding based on a change in measured impedance. The device 12 can also report temperature, heat flux, vasodilation and blood pressure along with the BI information. In one embodiment, the BI system monitors cardiac function using impedance cardiography (ICG) technique. ICG provides a single impedance tracing, from which parameters related to the pump function of the heart, such as cardiac output (CO), are estimated. ICG measures the beat-to-beat changes of thoracic bioimpedance via four dual sensors applied on the neck and thorax in order to calculate stroke volume (SV). More details are disclosed in U.S. Pat. No. 8,764,651 to the instant inventor, the content of which is incorporated by reference.

The impedance cardiographic embodiment allows hemodynamic assessment to be regularly monitored to avoid the occurrence of an acute cardiac episode. The system provides an accurate, noninvasive measurement of cardiac output (CO) monitoring so that ill and surgical animal or wearers undergoing major operations such as coronary artery bypass graft (CABG) would benefit. In addition, many animal or wearers with chronic and comorbid diseases that ultimately lead to the need for major operations and other costly interventions might benefit from more routine monitoring of CO and its dependent parameters such as systemic vascular resistance (SVR). Once SV has been determined, CO can be determined according to the following expression: CO=SV*HR, where HR=heart rate. CO can be determined for every heart-beat. Thus, the system can determine SV and CO on a beat-to-beat basis.

In one embodiment to monitor heart failure, an array of BI sensors are place in proximity to the heart. The array of BI sensors detect the presence or absence, or rate of change, or body fluids proximal to the heart. The BI sensors can be supplemented by the EKG sensors. A normal, healthy, heart beats at a regular rate. Irregular heart beats, known as cardiac arrhythmia, on the other hand, may characterize an unhealthy condition. Another unhealthy condition is known as congestive heart failure (“CHF”). CHF, also known as heart failure, is a condition where the heart has inadequate capacity to pump sufficient blood to meet metabolic demand. CHF may be caused by a variety of sources, including, coronary artery disease, myocardial infarction, high blood pressure, heart valve disease, cardiomyopathy, congenital heart disease, endocarditis, myocarditis, and others. Unhealthy heart conditions may be treated using a cardiac rhythm management (CRM) system. Examples of CRM systems, or pulse generator systems, include defibrillators (including implantable cardioverter defibrillator), pacemakers and other cardiac resynchronization devices.

In one implementation, BIA measurements can be made using an array of bipolar or tetrapolar electrodes that deliver a constant alternating current at 50 KHz frequency. Whole body measurements can be done using standard right-sided. The ability of any biological tissue to resist a constant electric current depends on the relative proportions of water and electrolytes it contains, and is called resistivity (in Ohms/cm3). The measuring of bioimpedance to assess congestive heart failure employs the different bio-electric properties of blood and lung tissue to permit separate assessment of: (a) systemic venous congestion via a low frequency or direct current resistance measurement of the current path through the right ventricle, right atrium, superior vena cava, and subclavian vein, or by computing the real component of impedance at a high frequency, and (b) pulmonary congestion via a high frequency measurement of capacitive impedance of the lung. The resistance is impedance measured using direct current or alternating current (AC) which can flow through capacitors.

In one embodiment, an array of noninvasive thoracic electrical bioimpedance monitoring probes can be used alone or in conjunction with other techniques such as impedance cardiography (ICG) for early comprehensive cardiovascular assessment and trending of acute trauma victims. This embodiment provides early, continuous cardiovascular assessment to help identify animal or wearers whose injuries were so severe that they were not likely to survive. This included severe blood and/or fluid volume deficits induced by trauma, which did not respond readily to expeditious volume resuscitation and vasopressor therapy. One exemplary system monitors cardiorespiratory variables that served as statistically significant measures of treatment outcomes: Qt, BP, pulse oximetry, and transcutaneous Po2 (Ptco2). A high Qt may not be sustainable in the presence of hypovolemia, acute anemia, pre-existing impaired cardiac function, acute myocardial injury, or coronary ischemia. Thus a fall in Ptco2 could also be interpreted as too high a metabolic demand for a animal or wearer's cardiovascular reserve. Too high a metabolic demand may compromise other critical organs. Acute lung injury from hypotension, blunt trauma, and massive fluid resuscitation can drastically reduce respiratory reserve.

One embodiment that measures thoracic impedance (a resistive or reactive impedance associated with at least a portion of a thorax of a living organism). The thoracic impedance signal is influenced by the animal or wearer's thoracic intravascular fluid tension, heart beat, and breathing (also referred to as “respiration” or “ventilation”). A “de” or “baseline” or “low frequency” component of the thoracic impedance signal (e.g., less than a cutoff value that is approximately between 0.1 Hz and 0.5 Hz, inclusive, such as, for example, a cutoff value of approximately 0.1 Hz) provides information about the subject animal or wearer's thoracic fluid tension, and is therefore influenced by intravascular fluid shifts to and away from the thorax. Higher frequency components of the thoracic impedance signal are influenced by the animal or wearer's breathing (e.g., approximately between 0.05 Hz and 2.0 Hz inclusive) and heartbeat (e.g., approximately between 0.5 Hz and 10 Hz inclusive). A low intravascular fluid tension in the thorax (“thoracic hypotension”) may result from changes in posture. For example, in a person who has been in a recumbent position for some time, approximately 1/3 of the blood volume is in the thorax. When that person then sits upright, approximately 1/3 of the blood that was in the thorax migrates to the lower body. This increases thoracic impedance. Approximately 90% of this fluid shift takes place within 2 to 3 minutes after the person sits upright.

The accelerometer can be used to provide reproducible measurements. Body activity will increase cardiac output and also change the amount of blood in the systemic venous system or lungs. Measurements of congestion may be most reproducible when body activity is at a minimum and the animal or wearer is at rest. The use of an accelerometer allows one to sense both body position and body activity. Comparative measurements over time may best be taken under reproducible conditions of body position and activity. Ideally, measurements for the upright position should be compared as among themselves. Likewise measurements in the supine, prone, left lateral decubitus and right lateral decubitus should be compared as among themselves. Other variables can be used to permit reproducible measurements, i.e. variations of the cardiac cycle and variations in the respiratory cycle. The ventricles are at their most compliant during diastole. The end of the diastolic period is marked by the QRS on the electrocardiographic means (EKG) for monitoring the cardiac cycle. The second variable is respiratory variation in impedance, which is used to monitor respiratory rate and volume. As the lungs fill with air during inspiration, impedance increases, and during expiration, impedance decreases. Impedance can be measured during expiration to minimize the effect of breathing on central systemic venous volume. While respiration and CHF both cause variations in impedance, the rates and magnitudes of the impedance variation are different enough to separate out the respiratory variations which have a frequency of about 8 to 60 cycles per minute and congestion changes which take at least several minutes to hours or even days to occur. Also, the magnitude of impedance change is likely to be much greater for congestive changes than for normal respiratory variation. Thus, the system can detect congestive heart failure (CHF) in early stages and alert a animal or wearer to prevent disabling and even lethal episodes of CHF. Early treatment can avert progression of the disorder to a dangerous stage.

In an embodiment to monitor wounds such as diabetic related wounds, the conductivity of a region of the animal or wearer with a wound or is susceptible to wound formation is monitored by the system. The system determines healing wounds if the impedance and reactance of the wound region increases as the skin region becomes dry. The system detects infected, open, interrupted healing, or draining wounds through lower regional electric impedances. In yet another embodiment, the bioimpedance sensor can be used to determine body fat. In one embodiment, the BI system determines Total Body Water (TBW) which is an estimate of total hydration level, including intracellular and extracellular water; Intracellular Water (ICW) which is an estimate of the water in active tissue and as a percent of a normal range (near 60% of TBW); Extracellular Water (ECW) which is water in tissues and plasma and as a percent of a normal range (near 40% of TBW); Body Cell Mass (BCM) which is an estimate of total pounds/kg of all active cells; Extracellular Tissue (ECT)/Extracellular Mass (ECM) which is an estimate of the mass of all other non-muscle inactive tissues including ligaments, bone and ECW; Fat Free Mass (FFM)/Lean Body Mass (LBM) which is an estimate of the entire mass that is not fat. It should be available in pounds/kg and may be presented as a percent with a normal range; Fat Mass (FM) which is an estimate of pounds/kg of body fat and percentage body fat; and Phase Angle (PA) which is associated with both nutrition and physical fitness.

Additional sensors such as thermocouples or thermisters and/or heat flux sensors can also be provided to provide measured values useful in analysis. In general, skin surface temperature will change with changes in blood flow in the vicinity of the skin surface of an organism. Such changes in blood flow can occur for a number of reasons, including thermal regulation, conservation of blood volume, and hormonal changes. In one implementation, skin surface measurements of temperature or heat flux are made in conjunction with hydration monitoring so that such changes in blood flow can be detected and appropriately treated.

In another embodiment, the device includes a Galvanic Skin Response (GSR) sensor. In this sensor, a small current is passed through one of the electrodes into the user's body such as the fingers and the CPU calculates how long it takes for a capacitor to fill up. The length of time the capacitor takes to fill up allows us to calculate the skin resistance: a short time means low resistance while a long time means high resistance. The GSR reflects sweat gland activity and changes in the sympathetic nervous system and measurement variables. Measured from the palm or fingertips, there are changes in the relative conductance of a small electrical current between the electrodes. The activity of the sweat glands in response to sympathetic nervous stimulation (Increased sympathetic activation) results in an increase in the level of conductance. Fear, anger, startle response, orienting response and sexual feelings are all among the emotions which may produce similar GSR responses.

In yet another embodiment, measurement of lung function such as peak expiratory flow readings is done though a sensor such as Wright's peak flow meter. In another embodiment, a respiratory estimator is provided that avoids the inconvenience of having the animal or wearer breathing through the flow sensor. In the respiratory estimator embodiment, heart period data from EKG/ECG is used to extract respiratory detection features. The heart period data is transformed into time-frequency distribution by applying a time-frequency transformation such as short-term Fourier transformation (STFT). Other possible methods are, for example, complex demodulation and wavelet transformation. Next, one or more respiratory detection features may be determined by setting up amplitude modulation of time-frequency plane, among others. The respiratory recognizer first generates a math model that correlates the respiratory detection features with the actual flow readings. The math model can be adaptive based on pre-determined data and on the combination of different features to provide a single estimate of the respiration. The estimator can be based on different mathematical functions, such as a curve fitting approach with linear or polynomical equations, and other types of neural network implementations, non-linear models, fuzzy systems, time series models, and other types of multivariate models capable of transferring and combining the information from several inputs into one estimate. Once the math model has been generated, the respirator estimator provides a real-time flow estimate by receiving EKG/ECG information and applying the information to the math model to compute the respiratory rate. Next, the computation of ventilation uses information on the tidal volume. An estimate of the tidal volume may be derived by utilizing different forms of information on the basis of the heart period signal. For example, the functional organization of the respiratory system has an impact in both respiratory period and tidal volume. Therefore, given the known relationships between the respiratory period and tidal volume during and transitions to different states, the information inherent in the heart period derived respiratory frequency may be used in providing values of tidal volume. In specific, the tidal volume contains inherent dynamics which may be, after modeling, applied to capture more closely the behavioral dynamics of the tidal volume. Moreover, it appears that the heart period signal, itself, is closely associated with tidal volume and may be therefore used to increase the reliability of deriving information on tidal volume. The accuracy of the tidal volume estimation may be further enhanced by using information on the subjects vital capacity (i.e., the maximal quantity of air that can be contained in the lungs during one breath). The information on vital capacity, as based on physiological measurement or on estimates derived from body measures such as height and weight, may be helpful in estimating tidal volume, since it is likely to reduce the effects of individual differences on the estimated tidal volume. Using information on the vital capacity, the mathematical model may first give values on the percentage of lung capacity in use, which may be then transformed to liters per breath. The optimizing of tidal volume estimation can based on, for example, least squares or other type of fit between the features and actual tidal volume. The minute ventilation may be derived by multiplying respiratory rate (breaths/min) with tidal volume (liters/breath).

In another embodiment, inductive plethysmography can be used to measure a cross-sectional area of the body by determining the self-inductance of a flexible conductor closely encircling the area to be measured. Since the inductance of a substantially planar conductive loop is well known to vary as, inter alia, the cross-sectional area of the loop, a inductance measurement may be converted into a plethysmographic area determination. Varying loop inductance may be measured by techniques known in the art, such as, e.g., by connecting the loop as the inductance in a variable frequency LC oscillator, the frequency of the oscillator then varying with the cross-sectional area of the loop inductance varies. Oscillator frequency is converted into a digital value, which is then further processed to yield the physiological parameters of interest. Specifically, a flexible conductor measuring a cross-sectional area of the body is closely looped around the area of the body so that the inductance, and the changes in inductance, being measured results from magnetic flux through the cross-sectional area being measured. The inductance thus depends directly on the cross-sectional area being measured, and not indirectly on an area which changes as a result of the factors changing the measured cross-sectional area. Various physiological parameters of medical and research interest may be extracted from repetitive measurements of the areas of various cross-sections of the body. For example, pulmonary function parameters, such as respiration volumes and rates and apneas and their types, may be determined from measurements of, at least, a chest transverse cross-sectional area and also an abdominal transverse cross-sectional area. Cardiac parameters, such central venous pressure, left and right ventricular volumes waveforms, and aortic and carotid artery pressure waveforms, may be extracted from repetitive measurements of transverse cross-sectional areas of the neck and of the chest passing through the heart. Timing measurements can be obtained from concurrent ECG measurements, and less preferably from the carotid pulse signal present in the neck. From the cardiac-related signals, indications of ischemia may be obtained independently of any ECG changes. Ventricular wall ischemia is known to result in paradoxical wall motion during ventricular contraction (the ischemic segment paradoxically “balloons” outward instead of normally contracting inward). Such paradoxical wall motion, and thus indications of cardiac ischemia, may be extracted from chest transverse cross-section area measurements. Left or right ventricular ischemia may be distinguished where paradoxical motion is seen predominantly in left or right ventricular waveforms, respectively. For another example, observations of the onset of contraction in the left and right ventricles separately may be of use in providing feedback to bi-ventricular cardiac pacing devices. For a further example, pulse oximetry determines hemoglobin saturation by measuring the changing infrared optical properties of a finger. This signal may be disambiguated and combined with pulmonary data to yield improved information concerning lung function.

In one embodiment to monitor and predict stroke attack, a cranial bioimpedance sensor is applied to detect fluids in the brain. The brain tissue can be modeled as an electrical circuit where cells with the lipid bilayer act as capacitors and the intra and extra cellular fluids act as resistors. The opposition to the flow of the electrical current through the cellular fluids is resistance. The system takes 50-kHz single-frequency bioimpedance measurements reflecting the electrical conductivity of brain tissue. The opposition to the flow of the current by the capacitance of lipid bilayer is reactance. In this embodiment, microamps of current at 50 kHz are applied to the electrode system. In one implementation, the electrode system consists of a pair of coaxial electrodes each of which has a current electrode and a voltage sensing electrode. For the measurement of cerebral bioimpedance, one pair of gel current electrodes is placed on closed eyelids and the second pair of voltage electrodes is placed in the suboccipital region projecting towards the foramen magnum. The electrical current passes through the orbital fissures and brain tissue. The drop in voltage is detected by the suboccipital electrodes and then calculated by the processor to bioimpedance values. The bioimpedance value is used to detect brain edema, which is defined as an increase in the water content of cerebral tissue which then leads to an increase in overall brain mass. Two types of brain edema are vasogenic or cytotoxic. Vasogenic edema is a result of increased capillary permeability. Cytotoxic edema reflects the increase of brain water due to an osmotic imbalance between plasma and the brain extracellular fluid. Cerebral edema in brain swelling contributes to the increase in intracranial pressure and an early detection leads to timely stroke intervention.

In another example, a cranial bioimpedance tomography system constructs brain impedance maps from surface measurements using nonlinear optimization. A nonlinear optimization technique utilizing known and stored constraint values permits reconstruction of a wide range of conductivity values in the tissue. In the nonlinear system, a Jacobian Matrix is renewed for a plurality of iterations. The Jacobian Matrix describes changes in surface voltage that result from changes in conductivity. The Jacobian Matrix stores information relating to the pattern and position of measuring electrodes, and the geometry and conductivity distributions of measurements resulting in a normal case and in an abnormal case. The nonlinear estimation determines the maximum voltage difference in the normal and abnormal cases.

In one embodiment, an electrode array sensor can include impedance, bio-potential, or electromagnetic field tomography imaging of cranial tissue. The electrode array sensor can be a geometric array of discrete electrodes having an equally-spaced geometry of multiple nodes that are capable of functioning as sense and reference electrodes. In a typical tomography application the electrodes are equally-spaced in a circular configuration. Alternatively, the electrodes can have non-equal spacing and/or can be in rectangular or other configurations in one circuit or multiple circuits. Electrodes can be configured in concentric layers too. Points of extension form multiple nodes that are capable of functioning as an electrical reference. Data from the multiple reference points can be collected to generate a spectrographic composite for monitoring over time.

The animal or wearer's brain cell generates an electromagnetic field of positive or negative polarity, typically in the millivolt range. The sensor measures the electromagnetic field by detecting the difference in potential between one or more test electrodes and a reference electrode. The bio-potential sensor uses signal conditioners or processors to condition the potential signal. In one example, the test electrode and reference electrode are coupled to a signal conditioner/processor that includes a lowpass filter to remove undesired high frequency signal components. The electromagnetic field signal is typically a slowly varying DC voltage signal. The lowpass filter removes undesired alternating current components arising from static discharge, electromagnetic interference, and other sources.

In one embodiment, the impedance sensor has an electrode structure with annular concentric circles including a central electrode, an intermediate electrode and an outer electrode, all of which are connected to the skin. One electrode is a common electrode and supplies a low frequency signal between this common electrode and another of the three electrodes. An amplifier converts the resulting current into a voltage between the common electrode and another of the three electrodes. A switch switches between a first circuit using the intermediate electrode as the common electrode and a second circuit that uses the outer electrode as a common electrode. The sensor selects depth by controlling the extension of the electric field in the vicinity of the measuring electrodes using the control electrode between the measuring electrodes. The control electrode is actively driven with the same frequency as the measuring electrodes to a signal level taken from one of the measuring electrodes but multiplied by a complex number with real and imaginary parts controlled to attain a desired depth penetration. The controlling field functions in the manner of a field effect transistor in which ionic and polarization effects act upon tissue in the manner of a semiconductor material.

With multiple groups of electrodes and a capability to measure at a plurality of depths, the system can perform tomographic imaging or measurement, and/or object recognition. In one embodiment, a fast reconstruction technique is used to reduce computation load by utilizing prior information of normal and abnormal tissue conductivity characteristics to estimate tissue condition without requiring full computation of a non-linear inverse solution.

In another embodiment, the bioimpedance system can be used with electro-encephalograph (EEG) or ERP. Since this embodiment collects signals related to blood flow in the brain, collection can be concentrated in those regions of the brain surface corresponding to blood vessels of interest. A headcap with additional electrodes placed in proximity to regions of the brain surface fed by a blood vessel of interest, such as the medial cerebral artery enables targeted information from the regions of interest to be collected. The headcap can cover the region of the brain surface that is fed by the medial cerebral artery. Other embodiments of the headcap can concentrate electrodes on other regions of the brain surface, such as the region associated with the somatosensory motor cortex. In alternative embodiments, the headcap can cover the skull more completely. Further, such a headcap can include electrodes thoughout the cap while concentrating electrodes in a region of interest. Depending upon the particular application, arrays of 1-16 head electrodes may be used, as compared to the International 10/20 system of 19-21 head electrodes generally used in an EEG instrument.

In one implementation, each amplifier for each EEG channel is a high quality analog amplifier device. Full bandwidth and ultra-low noise amplification are obtained for each electrode. Low pass, high pass, hum notch filters, gain, un-block, calibration and electrode impedance check facilities are included in each amplifier. All 8 channels in one EEG amplifier unit have the same filter, gain, etc. settings. Noise figures of less than 0.1 uV r.m.s. are achieved at the input and optical coupling stages. These figures, coupled with good isolation/common mode rejection result in signal clarity. Nine high pass filter ranges include 0.01 Hz for readiness potential measurement, and 30 Hz for EMG measurement.

In one embodiment, stimulations to elicit EEG signals are used in two different modes, i.e., auditory clicks and electric pulses to the skin. The stimuli, although concurrent, are at different prime number frequencies to permit separation of different evoked potentials (EPs) and avoid interference. Such concurrent stimulations for EP permit a more rapid, and less costly, examination and provide the animal or wearer's responses more quickly. Power spectra of spontaneous EEG, waveshapes of Averaged Evoked Potentials, and extracted measures, such as frequency specific power ratios, can be transmitted to a remote receiver. The latencies of successive EP peaks of the animal or wearer may be compared to those of a normal group by use of a normative template. To test for ischemic stroke or intracerebral or subarachnoid hemorrhage, the system provides a blood oxygen saturation monitor, using an infra-red or laser source, to alert the user if the animal or wearer's blood in the brain or some brain region is deoxygenated.

A stimulus device may optionally be placed on each subject, such as an audio generator in the form of an ear plug, which produces a series of “click” sounds. The subject's brain waves are detected and converted into audio tones. The device may have an array of LED (Light Emitting Diodes) which blink depending on the power and frequency composition of the brain wave signal. Power ratios in the frequencies of audio or somatosensory stimuli are similarly encoded. The EEG can be transmitted to a remote physician or medical aide who is properly trained to determine whether the animal or wearer's brain function is abnormal and may evaluate the functional state of various levels of the animal or wearer's nervous system.

The cranial bioimpedance sensor can be applied singly or in combination with a cranial blood flow sensor, which can be optical, ultrasound, electromagnetic sensor(s) as described in more details below. In an ultrasound imaging implementation, the carotid artery is checked for plaque build-up. Atherosclerosis is systemic—meaning that if the carotid artery has plaque buildup, other important arteries, such as coronary and leg arteries, might also be atherosclerotic.

In another embodiment, an epicardial array monopolar ECG system converts signals into the multichannel spectrum domain and identifies decision variables from the autospectra. The system detects and localizes the epicardial projections of ischemic myocardial ECGs during the cardiac activation phase. This is done by transforming ECG signals from an epicardial or torso sensor array into the multichannel spectral domain and identifying any one or more of a plurality of decision variables. The ECG array data can be used to detect, localize and quantify reversible myocardial ischemia.

In yet another embodiment, a trans-cranial Doppler velocimetry sensor provides a non-invasive technique for measuring blood flow in the brain. An ultrasound beam from a transducer is directed through one of three natural acoustical windows in the skull to produce a waveform of blood flow in the arteries using Doppler sonography. The data collected to determine the blood flow may include values such as the pulse cycle, blood flow velocity, end diastolic velocity, peak systolic velocity, mean flow velocity, total volume of cerebral blood flow, flow acceleration, the mean blood pressure in an artery, and the pulsatility index, or impedance to flow through a vessel. From this data, the condition of an artery may be derived, those conditions including stenosis, vasoconstriction, irreversible stenosis, vasodilation, compensatory vasodilation, hyperemic vasodilation, vascular failure, compliance, breakthrough, and pseudo-normalization.

In addition to the above techniques to detect stroke attack, the system can detect numbness or weakness of the face, arm or leg, especially on one side of the body. The system detects sudden confusion, trouble speaking or understanding, sudden trouble seeing in one or both eyes, sudden trouble walking, dizziness, loss of balance or coordination, or sudden, severe headache with no known cause.

In one embodiment to detect heart attack, the system detects discomfort in the center of the chest that lasts more than a few minutes, or that goes away and comes back. Symptoms can include pain or discomfort in one or both arms, the back, neck, jaw or stomach. The system can also monitor for shortness of breath which may occur with or without chest discomfort. Other signs may include breaking out in a cold sweat, nausea or lightheadedness.

The automated analyzer can also consider related pathologies in analyzing a animal or wearer's risk of stroke, including but not limited to gastritis, increased intracranial pressure, sleep disorders, small vessel disease, and vasculitis.

Other sensors can be used, for example devices for sensing EMG, EKG, blood pressure, sugar level, weight, temperature and pressure, among others. In one embodiment, an optical temperature sensor can be used. In another embodiment, a temperature thermistor can be used to sense animal or wearer temperature. In another embodiment, a fat scale sensor can be used to detect the animal or wearer's fat content. In yet another embodiment, a pressure sensor such as a MEMS sensor can be used to sense pressure on the animal or wearer.

In one embodiment, the sensors are mounted on the animal for detecting the body's electrical signals emanating from muscles (EMG and EOG) and brain (EEG) and cardiovascular system (ECG). Leg sensors can include piezoelectric accelerometers designed to give qualitative assessment of limb movement. Additionally, thoracic and abdominal bands used to measure expansion and contraction of the thorax and abdomen respectively. A small sensor can be mounted on the subject's finger in order to detect blood-oxygen levels and pulse rate. Additionally, a microphone can be attached to throat and used in sleep diagnostic recordings for detecting breathing and other noise. One or more position sensors can be used for detecting orientation of body (lying on left side, right side or back) during sleep diagnostic recordings. Each of sensors can individually transmit data to the server 20 using wired or wireless transmission. Alternatively, all sensors can be fed through a common bus into a single transceiver for wired or wireless transmission. The transmission can be done using a magnetic medium such as a floppy disk or a flash memory card, or can be done using infrared or

In one EKG or ECG detector, the heartbeat detection circuitry includes a differential amplifier for amplifying the signal transmitted from the EKG/ECG electrodes and for converting it into single-ended form, and a bandpass filter and a 60 Hz notch filter for removing background noise. The CPU measures the time durations between the successive pulses and estimates the heartbeat rate. The time durations between the successive pulses of the pulse sequence signal provides an estimate of heartbeat rate. Each time duration measurement is first converted to a corresponding rate, preferably expressed in beats per minute (bpm), and then stored in a file, taking the place of the earliest measurement previously stored. After a new measurement is entered into the file, the stored measurements are averaged, to produce an average rate measurement. The CPU optionally determines which of the stored measurements differs most from the average, and replaces that measurement with the average.

Upon initiation, the CPU increments a period timer used in measuring the time duration between successive pulses. This timer is incremented in steps of about two milliseconds in one embodiment. It is then determined whether or not a pulse has occurred during the previous two milliseconds. If it has not, the CPU returns to the initial step of incrementing the period timer. If a heartbeat has occurred, on the other hand, the CPU converts the time duration measurement currently stored in the period timer to a corresponding heartbeat rate, preferably expressed in bpm. After the heartbeat rate measurement is computed, the CPU determines whether or not the computed rate is intermediate prescribed thresholds of 20 bpm and 240 bpm. If it is not, it is assumed that the detected pulse was not in fact a heartbeat and the period timer is cleared.

In an optical heartbeat detector embodiment, an optical transducer generates a pulse oximeter waveform which is then analyzed to extract the beat-to-beat amplitude, area, and width (half height) measurements. The oximeter waveform is used to generate heartbeat rate in this embodiment. In one implementation, a reflective sensor such as the Honeywell HLC1395 can be used. The device emits lights from a window in the infrared spectrum and receives reflected light in a second window. When the heart beats, blood flow increases temporarily and more red blood cells flow through the windows, which increases the light reflected back to the detector. The light can be reflected, refracted, scattered, and absorbed by one or more detectors. Suitable noise reduction is done, and the resulting optical waveform is captured by the CPU.

In another optical embodiment, blood pressure is estimated from the optical reading using a mathematical model such as a linear correlation with a known blood pressure reading. In this embodiment, the pulse oximeter readings are compared to the blood-pressure readings from a known working blood pressure measurement device during calibration. Using these measurements, the linear equation is developed relating oximeter output waveform such as width to blood-pressure (systolic, mean and pulse pressure). In one embodiment, a transform (such as a Fourier analysis or a Wavelet transform) of the oximeter output can be used to generate a model to relate the oximeter output waveform to the blood pressure. Other non-linear math model or relationship can be determined to relate the oximeter waveform to the blood pressure.

In one embodiment, to determine blood flow velocity, acoustic pulses are generated and transmitted into the artery using an ultrasonic transducer positioned near an artery. These pulses are reflected by various structures or entities within the artery (such as the artery walls, and the red blood cells within the subject's blood), and subsequently received as frequency shifts by the ultrasonic transducer. Next, the blood flow velocity is determined. In this process, the frequencies of those echoes reflected by blood cells within the blood flowing in the artery differ from that of the transmitted acoustic pulses due to the motion of the blood cells. This “Doppler shift” in frequency is used to calculate the blood flow velocity. In one embodiment for determining blood flow velocity, the Doppler frequency is used to determine mean blood velocity. For example, U.S. Pat. No. 6,514,211, the content of which is incorporated by reference, discusses blood flow velocity using a time-frequency representation.

In one implementation, the system can obtain one or more numerical calibration curves describing the animal or wearer's vital signs such as blood pressure. The system can then direct energy such as infrared or ultrasound at the animal or wearer's artery and detecting reflections thereof to determine blood flow velocity from the detected reflections. The system can numerically fit or map the blood flow velocity to one or more calibration parameters describing a vital-sign value. The calibration parameters can then be compared with one or more numerical calibration curves to determine the blood pressure.

Additionally, the system can analyze blood pressure, and heart rate, and pulse oximetry values to characterize the user's cardiac condition. These programs, for example, may provide a report that features statistical analysis of these data to determine averages, data displayed in a graphical format, trends, and comparisons to doctor-recommended values.

In one embodiment, feed forward artificial neural networks (NNs) are used to classify valve-related heart disorders. The heart sounds are captured using the microphone or piezoelectric transducer. Relevant features were extracted using several signal processing tools, discrete wavelet transfer, fast fourier transform, and linear prediction coding. The heart beat sounds are processed to extract the necessary features by: a) denoising using wavelet analysis, b) separating one beat out of each record c) identifying each of the first heart sound (FHS) and the second heart sound (SHS). Valve problems are classified according to the time separation between the FHS and the SHS relative to cardiac cycle time, namely whether it is greater or smaller than 20% of cardiac cycle time. In one embodiment, the NN comprises 6 nodes at both ends, with one hidden layer containing 10 nodes. In another embodiment, linear predictive code (LPC) coefficients for each event were fed to two separate neural networks containing hidden neurons.

In another embodiment, a normalized energy spectrum of the sound data is obtained by applying a Fast Fourier Transform. The various spectral resolutions and frequency ranges were used as inputs into the NN to optimize these parameters to obtain the most favorable results.

In another embodiment, the heart sounds are denoised using six-stage wavelet decomposition, thresholding, and then reconstruction. Three feature extraction techniques were used: the Decimation method, and the wavelet method. Classification of the heart diseases is done using Hidden Markov Models (HMMs).

In yet another embodiment, a wavelet transform is applied to a window of two periods of heart sounds. Two analyses are realized for the signals in the window: segmentation of first and second heart sounds, and the extraction of the features. After segmentation, feature vectors are formed by using the wavelet detail coefficients at the sixth decomposition level. The best feature elements are analyzed by using dynamic programming.

In another embodiment, the wavelet decomposition and reconstruction method extract features from the heart sound recordings. An artificial neural network classification method classifies the heart sound signals into physiological and pathological murmurs. The heart sounds are segmented into four parts: the first heart sound, the systolic period, the second heart sound, and the diastolic period. The following features can be extracted and used in the classification algorithm: a) Peak intensity, peak timing, and the duration of the first heart sound b) the duration of the second heart sound c) peak intensity of the aortic component of S2(A2) and the pulmonic component of S2 (P2), the splitting interval and the reverse flag of A2 and P2, and the timing of A2 d) the duration, the three largest frequency components of the systolic signal and the shape of the envelope of systolic murmur e) the duration the three largest frequency components of the diastolic signal and the shape of the envelope of the diastolic murmur.

In one embodiment, the time intervals between the ECG R-waves are detected using an envelope detection process. The intervals between R and T waves are also determined. The Fourier transform is applied to the sound to detect S1 and S2. To expedite processing, the system applies Fourier transform to detect S1 in the interval 0.1-0.5 R-R. The system looks for S2 the intervals R-T and 0.6 R-R. S2 has an aortic component A2 and a pulmonary component P2. The interval between these two components and its changes with respiration has clinical significance. A2 sound occurs before P2, and the intensity of each component depends on the closing pressure and hence A2 is louder than P2. The third heard sound S3 results from the sudden halt in the movement of the ventricle in response to filling in early diastole after the AV valves and is normally observed in children and young adults. The fourth heart sound S4 is caused by the sudden halt of the ventricle in response to filling in presystole due to atrial contraction.

In yet another embodiment, the S2 is identified and a normalized splitting interval between A2 and P2 is determined. If there is no overlap, A2 and P2 are determined from the heart sound. When overlap exists between A2 and P2, the sound is dechirped for identification and extraction of A2 and P2 from S2. The A2-P2 splitting interval (S1) is calculated by computing the cross-correlation function between A2 and P2 and measuring the time of occurrence of its maximum amplitude. SI is then normalized (NSI) for heart rate as follows: NSI=SI/cardiac cycle time. The duration of the cardiac cycle can be the average interval of QRS waves of the ECG. It could also be estimated by computing the mean interval between a series of consecutive S1 and S2 from the heart sound data. A non linear regressive analysis maps the relationship between the normalized NSI and PAP. A mapping process such as a curve-fitting procedure determines the curve that provides the best fit with the animal or wearer data. Once the mathematical relationship is determined, NSI can be used to provide an accurate quantitative estimate of the systolic and mean PAP relatively independent of heart rate and systemic arterial pressure.

In another embodiment, the first heart sound (S1) is detected using a time-delayed neural network (TDNN). The network consists of a single hidden layer, with time-delayed links connecting the hidden units to the time-frequency energy coefficients of a Morlet wavelet decomposition of the input phonocardiogram (PCG) signal. The neural network operates on a 200 msec sliding window with each time-delay hidden unit spanning 100 msec of wavelet data.

In yet another embodiment, a local signal analysis is used with a classifier to detect, characterize, and interpret sounds corresponding to symptoms important for cardiac diagnosis. The system detects a plurality of different heart conditions. Heart sounds are automatically segmented into a segment of a single heart beat cycle. Each segment are then transformed using 7 level wavelet decomposition, based on Coffman 4th order wavelet kernel. The resulting vectors 4096 values, are reduced to 256 element feature vectors, this simplified the neural network and reduced noise.

In another embodiment, feature vectors are formed by using the wavelet detail and approximation coefficients at the second and sixth decomposition levels. The classification (decision making) is performed in 4 steps: segmentation of the first and second heart sounds, normalization process, feature extraction, and classification by the artificial neural network.

In another embodiment using decision trees, the system distinguishes (1) the Aortic Stenosis (AS) from the Mitral Regurgitation (MR) and (2) the Opening Snap (OS), the Second Heart Sound Split (A2_P2) and the Third Heart Sound (S3). The heart sound signals are processed to detect the first and second heart sounds in the following steps: a) wavelet decomposition, b) calculation of normalized average Shannon Energy, c) a morphological transform action that amplifies the sharp peaks and attenuates the broad ones d) a method that selects and recovers the peaks corresponding to S1 and S2 and rejects others e) algorithm that determines the boundaries of S1 and S2 in each heart cycle f) a method that distinguishes 51 from S2.

In one embodiment, once the heart sound signal has been digitized and captured into the memory, the digitized heart sound signal is parameterized into acoustic features by a feature extractor. The output of the feature extractor is delivered to a sound recognizer. The feature extractor can include the short time energy, the zero crossing rates, the level crossing rates, the filter-bank spectrum, the linear predictive coding (LPC), and the fractal method of analysis. In addition, vector quantization may be utilized in combination with any representation techniques. Further, one skilled in the art may use an auditory signal-processing model in place of the spectral models to enhance the system's robustness to noise and reverberation

In one embodiment of the feature extractor, the digitized heart sound signal series s(n) is put through a low-order filter, typically a first-order finite impulse response filter, to spectrally flatten the signal and to make the signal less susceptible to finite precision effects encountered later in the signal processing. The signal is pre-emphasized preferably using a fixed pre-emphasis network, or preemphasizer. The signal can also be passed through a slowly adaptive pre-emphasizer. The preemphasized heart sound signal is next presented to a frame blocker to be blocked into frames of N samples with adjacent frames being separated by M samples. In one implementation, frame 1 contains the first 400 samples. The frame 2 also contains 400 samples, but begins at the 300th sample and continues until the 700th sample. Because the adjacent frames overlap, the resulting LPC spectral analysis will be correlated from frame to frame. Each frame is windowed to minimize signal discontinuities at the beginning and end of each frame. The windower tapers the signal to zero at the beginning and end of each frame. Preferably, the window used for the autocorrelation method of LPC is the Hamming window. A noise canceller operates in conjunction with the autocorrelator to minimize noise. Noise in the heart sound pattern is estimated during quiet periods, and the temporally stationary noise sources are damped by means of spectral subtraction, where the autocorrelation of a clean heart sound signal is obtained by subtracting the autocorrelation of noise from that of corrupted heart sound. In the noise cancellation unit, if the energy of the current frame exceeds a reference threshold level, the heart is generating sound and the autocorrelation of coefficients representing noise is not updated. However, if the energy of the current frame is below the reference threshold level, the effect of noise on the correlation coefficients is subtracted off in the spectral domain. The result is half-wave rectified with proper threshold setting and then converted to the desired autocorrelation coefficients. The output of the autocorrelator and the noise canceller are presented to one or more parameterization units, including an LPC parameter unit, an FFT parameter unit, an auditory model parameter unit, a fractal parameter unit, or a wavelet parameter unit, among others. The LPC parameter is then converted into cepstral coefficients. The cepstral coefficients are the coefficients of the Fourier transform representation of the log magnitude spectrum. A filter bank spectral analysis, which uses the short-time Fourier transformation (STFT) may also be used alone or in conjunction with other parameter blocks. FFT is well known in the art of digital signal processing. Such a transform converts a time domain signal, measured as amplitude over time, into a frequency domain spectrum, which expresses the frequency content of the time domain signal as a number of different frequency bands. The FFT thus produces a vector of values corresponding to the energy amplitude in each of the frequency bands. The FFT converts the energy amplitude values into a logarithmic value which reduces subsequent computation since the logarithmic values are more simple to perform calculations on than the longer linear energy amplitude values produced by the FFT, while representing the same dynamic range. Ways for improving logarithmic conversions are well known in the art, one of the simplest being use of a look-up table. In addition, the FFT modifies its output to simplify computations based on the amplitude of a given frame. This modification is made by deriving an average value of the logarithms of the amplitudes for all bands. This average value is then subtracted from each of a predetermined group of logarithms, representative of a predetermined group of frequencies. The predetermined group consists of the logarithmic values, representing each of the frequency bands. Thus, utterances are converted from acoustic data to a sequence of vectors of k dimensions, each sequence of vectors identified as an acoustic frame, each frame represents a portion of the utterance. Alternatively, auditory modeling parameter unit can be used alone or in conjunction with others to improve the parameterization of heart sound signals in noisy and reverberant environments. In this approach, the filtering section may be represented by a plurality of filters equally spaced on a log-frequency scale from 0 Hz to about 3000 Hz and having a prescribed response corresponding to the cochlea. The nerve fiber firing mechanism is simulated by a multilevel crossing detector at the output of each cochlear filter. The ensemble of the multilevel crossing intervals corresponds to the firing activity at the auditory nerve fiber-array. The interval between each successive pair of same direction, either positive or negative going, crossings of each predetermined sound intensity level is determined and a count of the inverse of these interspike intervals of the multilevel detectors for each spectral portion is stored as a function of frequency. The resulting histogram of the ensemble of inverse interspike intervals forms a spectral pattern that is representative of the spectral distribution of the auditory neural response to the input sound and is relatively insensitive to noise. The use of a plurality of logarithmically related sound intensity levels accounts for the intensity of the input signal in a particular frequency range. Thus, a signal of a particular frequency having high intensity peaks results in a much larger count for that frequency than a low intensity signal of the same frequency. The multiple level histograms of the type described herein readily indicate the intensity levels of the nerve firing spectral distribution and cancel noise effects in the individual intensity level histograms. Alternatively, the fractal parameter block can further be used alone or in conjunction with others to represent spectral information. Fractals have the property of self similarity as the spatial scale is changed over many orders of magnitude. A fractal function includes both the basic form inherent in a shape and the statistical or random properties of the replacement of that shape in space. As is known in the art, a fractal generator employs mathematical operations known as local affine transformations. These transformations are employed in the process of encoding digital data representing spectral data. The encoded output constitutes a “fractal transform” of the spectral data and consists of coefficients of the affine transformations. Different fractal transforms correspond to different images or sounds.

Alternatively, a wavelet parameterization block can be used alone or in conjunction with others to generate the parameters. Like the FFT, the discrete wavelet transform (DWT) can be viewed as a rotation in function space, from the input space, or time domain, to a different domain. The DWT consists of applying a wavelet coefficient matrix hierarchically, first to the full data vector of length N, then to a smooth vector of length N/2, then to the smooth-smooth vector of length N/4, and so on. Most of the usefulness of wavelets rests on the fact that wavelet transforms can usefully be severely truncated, or turned into sparse expansions. In the DWT parameterization block, the wavelet transform of the heart sound signal is performed. The wavelet coefficients are allocated in a non-uniform, optimized manner. In general, large wavelet coefficients are quantized accurately, while small coefficients are quantized coarsely or even truncated completely to achieve the parameterization. Due to the sensitivity of the low-order cepstral coefficients to the overall spectral slope and the sensitivity of the high-order cepstral coefficients to noise variations, the parameters generated may be weighted by a parameter weighing block, which is a tapered window, so as to minimize these sensitivities. Next, a temporal derivator measures the dynamic changes in the spectra. Power features are also generated to enable the system to distinguish heart sound from silence.

After the feature extraction has been performed, the heart sound parameters are next assembled into a multidimensional vector and a large collection of such feature signal vectors can be used to generate a much smaller set of vector quantized (VQ) feature signals by a vector quantizer that cover the range of the larger collection. In addition to reducing the storage space, the VQ representation simplifies the computation for determining the similarity of spectral analysis vectors and reduces the similarity computation to a look-up table of similarities between pairs of codebook vectors. To reduce the quantization error and to increase the dynamic range and the precision of the vector quantizer, the preferred embodiment partitions the feature parameters into separate codebooks, preferably three. In the preferred embodiment, the first, second and third codebooks correspond to the cepstral coefficients, the differenced cepstral coefficients, and the differenced power coefficients.

With conventional vector quantization, an input vector is represented by the codeword closest to the input vector in terms of distortion. In conventional set theory, an object either belongs to or does not belong to a set. This is in contrast to fuzzy sets where the membership of an object to a set is not so clearly defined so that the object can be a part member of a set. Data are assigned to fuzzy sets based upon the degree of membership therein, which ranges from 0 (no membership) to 1.0 (full membership). A fuzzy set theory uses membership functions to determine the fuzzy set or sets to which a particular data value belongs and its degree of membership therein.

To handle the variance of heart sound patterns of individuals over time and to perform speaker adaptation in an automatic, self-organizing manner, an adaptive clustering technique called hierarchical spectral clustering is used. Such speaker changes can result from temporary or permanent changes in vocal tract characteristics or from environmental effects. Thus, the codebook performance is improved by collecting heart sound patterns over a long period of time to account for natural variations in speaker behavior. In one embodiment, data from the vector quantizer is presented to one or more recognition models, including an HMM model, a dynamic time warping model, a neural network, a fuzzy logic, or a template matcher, among others. These models may be used singly or in combination.

In dynamic processing, at the time of recognition, dynamic programming slides, or expands and contracts, an operating region, or window, relative to the frames of heart sound so as to align those frames with the node models of each S1-S4 pattern to find a relatively optimal time alignment between those frames and those nodes. The dynamic processing in effect calculates the probability that a given sequence of frames matches a given word model as a function of how well each such frame matches the node model with which it has been time-aligned. The word model which has the highest probability score is selected as corresponding to the heart sound.

Dynamic programming obtains a relatively optimal time alignment between the heart sound to be recognized and the nodes of each word model, which compensates for the unavoidable differences in speaking rates which occur in different utterances of the same word. In addition, since dynamic programming scores words as a function of the fit between word models and the heart sound over many frames, it usually gives the correct word the best score, even if the word has been slightly misspoken or obscured by background sound. This is important, because animals often mispronounce words either by deleting or mispronouncing proper sounds, or by inserting sounds which do not belong.

In dynamic time warping (DTW), the input heart sound A, defined as the sampled time values A=a(1) . . . a(n), and the vocabulary candidate B, defined as the sampled time values B=b(1) . . . b(n), are matched up to minimize the discrepancy in each matched pair of samples. Computing the warping function can be viewed as the process of finding the minimum cost path from the beginning to the end of the words, where the cost is a function of the discrepancy between the corresponding points of the two words to be compared. Dynamic programming considers all possible points within the permitted domain for each value of i. Because the best path from the current point to the next point is independent of what happens beyond that point. Thus, the total cost of [i(k), j(k)] is the cost of the point itself plus the cost of the minimum path to it. Preferably, the values of the predecessors can be kept in an M×N array, and the accumulated cost kept in a 2×N array to contain the accumulated costs of the immediately preceding column and the current column. However, this method requires significant computing resources. For the heart sound recognizer to find the optimal time alignment between a sequence of frames and a sequence of node models, it must compare most frames against a plurality of node models. One method of reducing the amount of computation required for dynamic programming is to use pruning Pruning terminates the dynamic programming of a given portion of heart sound against a given word model if the partial probability score for that comparison drops below a given threshold. This greatly reduces computation, since the dynamic programming of a given portion of heart sound against most words produces poor dynamic programming scores rather quickly, enabling most words to be pruned after only a small percent of their comparison has been performed. To reduce the computations involved, one embodiment limits the search to that within a legal path of the warping.

A Hidden Markov model can be used in one embodiment to evaluate the probability of occurrence of a sequence of observations O(1), O(2), . . . O(t), . . . , O(T), where each observation O(t) may be either a discrete symbol under the VQ approach or a continuous vector. The sequence of observations may be modeled as a probabilistic function of an underlying Markov chain having state transitions that are not directly observable. The transitions between states are represented by a transition matrix A=[a(i,j)]. Each a(i,j) term of the transition matrix is the probability of making a transition to state j given that the model is in state i. The output symbol probability of the model is represented by a set of functions B=[b(j)(O(t)], where the b(j)(O(t) term of the output symbol matrix is the probability of outputting observation O(t), given that the model is in state j. The first state is always constrained to be the initial state for the first time frame of the utterance, as only a prescribed set of left-to-right state transitions are possible. A predetermined final state is defined from which transitions to other states cannot occur. Transitions are restricted to reentry of a state or entry to one of the next two states. Such transitions are defined in the model as transition probabilities. For example, a heart sound pattern currently having a frame of feature signals in state 2 has a probability of reentering state 2 of a(2,2), a probability a(2,3) of entering state 3 and a probability of a(2,4)=1−a(2, 1)−a(2,2) of entering state 4. The probability a(2, 1) of entering state 1 or the probability a(2,5) of entering state 5 is zero and the sum of the probabilities a(2,1) through a(2,5) is one. Although the preferred embodiment restricts the flow graphs to the present state or to the next two states, one skilled in the art can build an HMM model without any transition restrictions.

The Markov model is formed for a reference pattern from a plurality of sequences of training patterns and the output symbol probabilities are multivariate Gaussian function probability densities. The heart sound traverses through the feature extractor. During learning, the resulting feature vector series is processed by a parameter estimator, whose output is provided to the hidden Markov model. The hidden Markov model is used to derive a set of reference pattern templates, each template representative of an identified S1-S4 pattern in a vocabulary set of reference patterns. The Markov model reference templates are next utilized to classify a sequence of observations into one of the reference patterns based on the probability of generating the observations from each Markov model reference pattern template. During recognition, the unknown pattern can then be identified as the reference pattern with the highest probability in the likelihood calculator.

In one embodiment, a heart sound analyzer detects Normal S1, Split S1, Normal S2, Normal split S2, Wide split S2, Paradoxical split S2, Fixed split S2, S3 right ventricle origin, S3 left ventricle origin, opening snap, S4 right ventricle origin, S4 left ventricle origin, aortic ejection sound, and pulmonic ejection sound, among others. The sound analyzer can be an HMM type analyzer, a neural network type analyzer, a fuzzy logic type analyzer, a genetic algorithm type analyzer, a rule-based analyzer, or any suitable classifier. The heart sound data is captured, filtered, and the major features of the heart sound are determined and then operated by a classifier such as HMM or neural network, among others.

The analyzer can detect S1, whose major audible components are related to mitral and tricuspid valve closure. Mitral (MI) closure is the first audible component of the first sound. It normally occurs before tricuspid (T1) closure, and is of slightly higher intensity than T1. A split of the first sound occurs when both components that make up the sound are separately distinguishable. In a normally split first sound, the mitral and tricuspid components are 20 to 30 milliseconds apart. Under certain conditions a wide or abnormally split first sound can be heard. An abnormally wide split first sound can be due to either electrical or mechanical causes, which create asynchrony of the two ventricles. Some of the electrical causes may be right bundle branch block, premature ventricular beats and ventricular tachycardia. An apparently wide split can be caused by another sound around the time of the first. The closure of the aortic and pulmonic valves contributes to second sound production. In the normal sequence, the aortic valve closes before the pulmonic valve. The left sided mechanical events normally precede right sided events.

The system can analyze the second sound S2. The aortic (A2) component of the second sound is the loudest of the two components and is discernible at all auscultation sites, but especially well at the base. The pulmonic (P2) component of the second sound is the softer of the two components and is usually audible at base left. A physiological split occurs when both components of the second sound are separately distinguishable. Normally this split sound is heard on inspiration and becomes single on expiration. The A2 and P2 components of the physiological split usually coincide, or are less than 30 milliseconds apart during expiration and often moved to around 50 to 60 milliseconds apart by the end of inspiration. The physiological split is heard during inspiration because it is during that respiratory cycle that intrathoracic pressure drops. This drop permits more blood to return to the right heart. The increased blood volume in the right ventricle results in a delayed pulmonic valve closure. At the same time, the capacity of the pulmonary vessels in the lung is increased, which results in a slight decrease in the blood volume returning to the left heart. With less blood in the left ventricle, its ejection takes less time, resulting in earlier closing of the aortic valve. Therefore, the net effect of inspiration is to cause aortic closure to occur earlier, and pulmonary closure to occur later. Thus, a split second is heard during inspiration, and a single second sound is heard during expiration. A reversed (paradoxical) split of the second sound occurs when there is a reversal of the normal closure sequence with pulmonic closure occurring before aortic. During inspiration the second sound is single, and during expiration the second sound splits. This paradoxical splitting of the second sound may be heard when aortic closure is delayed, as in marked volume or pressure loads on the left ventricle (i.e., aortic stenosis) or with conduction defects which delay left ventricular depolarization (i.e., left bundle branch block). The normal physiological split second sound can be accentuated by conditions that cause an abnormal delay in pulmonic valve-1 closure. Such a delay may be due to an increased volume in the right ventricle as o compared with the left (atrial septal defect, or ventricular septal defect); chronic right ventricular outflow obstruction (pulmonic stenosis); acute or chronic dilatation of the right ventricle due to sudden rise in pulmonary artery pressure (pulmonary embolism); electrical delay or activation of AA the right ventricle (right bundle branch block); decreased elastic recoil of the pulmonary artery (idiopathic dilatation of the pulmonary artery). The wide split has a duration of 40 to 50′ milliseconds, compared to the normal physiologic split of 30 milliseconds. Fixed splitting of the second sound refers to split sound which displays little or no respiratory variation. The two components making up the sound occur in their normal sequence, but the ventricles are unable to change their volumes with respiration. This finding is typical in atrial septal defect, but is occasionally heard in congestive heart failure. The fixed split is heard best at base left with the diaphragm.

The third heart sound is also of low frequency, but it is heard just after the second heart sound. It occurs in early diastole, during the time of rapid ventricular filling. This sound occurs about 140 to 160 milliseconds after the second sound. The S3 is often heard in normal children or young adults but when heard in individuals over the age of 40 it usually reflects cardiac disease characterized by ventricular dilatation, decreased systolic function, and elevated ventricular diastolic filling pressure. The nomenclature includes the term ventricular gallop, protodiastolic gallop, S3 gallop, or the more common, S3. When normal it is referred to as a physiological third heart sound, and is usually not heard past the age of forty. The abnormal, or pathological third heart sound, may be heard in individuals with coronary artery disease, cardiomyopathies, incompetent valves, left to right shunts, Ventricular Septal Defect (VSD), or Patent Ductus Arteriosus (PDA). The pathological S3 may be the first clinical sign of congestive heart failure. The fourth heart sound is a low frequency sound heard just before the first heart sound, usually preceding this sound by a longer interval than that separating the two components of the normal first sound. It has also been known as an “atrial gallop”, a “presystolic gallop”, and an “S4 gallop”. It is most commonly known as an “S4”.

The S4 is a diastolic sound, which occurs during the late diastolic filling phase at the time when the atria contract. When the ventricles have a decreased compliance, or are receiving an increased diastolic volume, they generate a low frequency vibration, the S4. Some authorities believe the S4 may be normal in youth, but is seldom considered normal after the age of 20. The abnormal or pathological S4 is heard in primary myocardial disease, coronary artery disease, hypertension, and aortic and pulmonic stenosis. The S4 may have its origin in either the left or right heart. The S4 of left ventricular origin is best heard at the apex, with the animal or wearer supine, or in the left lateral recumbent position. Its causes include severe hypertension, aortic stenosis, cardiomyopathies, and left ventricular myocardial infarctions. In association with ischemic heart disease the S4 is often loudest during episodes of angina pectoris or may occur early after an acute myocardial infarction, often becoming fainter as the animal or wearer improves. The S4 of right ventricular origin is best heard at the left lateral sternal border. It is usually accentuated with inspiration, and may be due to pulmonary stenosis, pulmonary hypertension, or right ventricular myocardial infarction. When both the third heart sound and a fourth heart sound are present, with a normal heart rate, 60-100 heart beats per minute, the four sound cadence of a quadruple rhythm may be heard.

Ejection sounds are high frequency clicky sounds occurring shortly after the first sound with the onset of ventricular ejection. They are produced by the opening of the semilunar valves, aortic or pulmonic, either when one of these valves is diseased, or when ejection is rapid through a normal valve. They are heard best at the base, and may be of either aortic or pulmonic origin. Ejection sounds of aortic origin often radiate widely and may be heard anywhere on a straight line from the base right to the apex. Aortic ejection sounds are most typically heard in animal or wearers with valvular aortic stenosis, but are occasionally heard in various other conditions, such as aortic insufficiency, coarctation of the aorta, or aneurysm of the ascending aorta. Ejection sounds of pulmonic origin are heard anywhere on a straight line from base left, where they are usually best heard, to the epigastrium Pulmonic ejection sounds are typically heard in pulmonic stenosis, but may be encountered in pulmonary hypertension, atrial septal defects (ASD) or in conditions causing enlargement of the pulmonary artery. Clicks are high frequency sounds which occur in systole, either mid, early, or late. The click generally occurs at least 100 milliseconds after the first sound. The most common cause of the click is mitral valve prolapse. The clicks of mitral origin are best heard at the apex, or toward the left lateral sternal border. The click will move closer to the first sound when volume to the ventricle is reduced, as occurs in standing or the Valsalva maneuver. The opening snap is a short high frequency sound, which occurs after the second heart sound in early diastole. It usually follows the second sound by about 60 to 100 milliseconds. It is most frequently the result of the sudden arrest of the opening of the mitral valve, occurring in mitral stenosis, but may also be encountered in conditions producing increased flow through this valve (i.e., VSD or PDA). In tricuspid stenosis or in association with increased flow across the tricuspid valve, as in ASD, a tricuspid opening snap may be heard. The tricuspid opening snap is loudest at the left lateral sternal border, and becomes louder with inspiration.

Murmurs are sustained noises that are audible during the time periods of systole, diastole, or both. They are basically produced by these factors: 1) Backward regurgitation through a leaking valve or septal defect; 2) Forward flow through a narrowed or deformed valve or conduit or through an arterial venous connection; 3) High rate of blood flow through a normal or abnormal valve; 4) Vibration of loose structures within the heart (i.e., chordae tendineae or valvular tissue). Murmurs that occur when the ventricles are contracting, that is, during systole, are referred to as systolic murmurs. Murmurs occurring when the ventricles are relaxed and filling, that is during diastole, are referred to as diastolic murmurs. There are six characteristics useful in murmur identification and differentiation:

1) Location or the valve area over which the murmur is best heard. This is one clue to the origin of the murmur. Murmurs of mitral origin are usually best heard at the apex. Tricuspid murmurs at the lower left lateral sternal border, and pulmonic murmurs at base left. Aortic systolic murmurs are best heard at base right, and aortic diastolic murmurs at Erb's point, the third intercostal space to the left of the sternum.

2) Frequency (pitch). Low, medium, or high.

3) Intensity.

4) Quality.

5) Timing. (Occurring during systole, diastole, or both).

6) Areas where the sound is audible in addition to the area over which it is heard best.

Systolic murmurs are sustained noises that are audible during the time period of systole, or the period between S1 and S2. Forward flow across the aortic or pulmonic valves, or regurgitant flow from the mitral or tricuspid valve may produce a systolic murmur. Systolic murmurs may be normal, and can represent normal blood flow, i.e., thin chest, babies and children, or increased blood flow, i.e., pregnant women. Early systolic murmurs begin with or shortly after the first sound and peak in the first third of systole. Early murmurs have the greatest intensity in the early part of the cycle. The commonest cause is the innocent murmur of childhood (to be discussed later). A small ventricular septal defect (VSD) occasionally causes an early systolic murmur. The early systolic murmur of a small VSD begins with S1 and stops in mid systole, because as ejection continues and the ventricular size decreases, the small defect is sealed shut, causing the murmur to soften or cease. This murmur is characteristic of the type of children's VSD located in the muscular portion of the ventricular septum. This defect may disappear with age. A mid-systolic murmur begins shortly after the first sound, peaks in the middle of systole, and does not quite extend to the second sound. It is the crescendo decrescendo murmur which builds up and decrease symmetrically. It is also known as an ejection murmur. It most commonly is due to forward blood flow through a normal, narrow or irregular valve, i.e., aortic or pulmonic stenosis. The murmur begins when the pressure in the respective ventricle exceeds the aortic or pulmonary arterial pressure. The most characteristic feature of this murmur is its cessation before the second sound, thus leaving this latter sound identifiable as a discrete entity. This type of murmur is commonly heard in normal individuals, particularly in the young, who usually have increased blood volumes flowing over normal valves. In this setting the murmur is usually short, with its peak intensity early in systole, and is soft, seldom over 2 over 6 in intensity. It is then designated as an innocent murmur. In order for a murmur to be classified as innocent (i.e. normal), the following are present:

1) Normal splitting of the second sound together with absence of abnormal sounds or murmurs, such as ejection sounds, diastolic murmurs, etc.

2) Normal jugular venus and carotid pulses

3) Normal precordial pulsations or palpation, and

4) Normal chest x-ray and ECG

Obstruction or stenosis across the aortic or pulmonic valves also may give rise to a murmur of this type. These murmurs are usually longer and louder than the innocent murmur, and reach a peak intensity in mid-systole. The murmur of aortic stenosis is harsh in quality and is heard equally well with either the bell or the diaphragm. It is heard best at base right, and radiates to the apex and to the neck bilaterally.

An early diastolic murmur begins with a second sound, and peaks in the first third of diastole. Common causes are aortic regurgitation and pulmonic regurgitation. The early diastolic murmur of aortic regurgitation usually has a high frequency blowing quality, is heard best with a diaphragm at Erb's point, and radiates downward along the left sternal border. Aortic regurgitation tends to be of short duration, and heard best on inspiration. This respiratory variation is helpful in differentiating pulmonic regurgitation from aortic regurgitation. A mid-diastolic murmur begins after the second sound and peaks in mid-diastole. Common causes are mitral stenosis, and tricuspid stenosis. The murmur of mitral stenosis is a low frequency, crescendo de crescendo rumble, heard at the apex with the bell lightly held. If it radiates, it does so minimally to the axilla. Mitral stenosis normally produces three distinct abnormalities which can be heard: 1) A loud first sound 2) An opening snap, and 3) A mid-diastolic rumble with a late diastolic accentuation.

A late diastolic murmur occurs in the latter half of diastole, synchronous with atrial contraction, and extends to the first sound. Although occasionally occurring alone, it is usually a component of the longer diastolic murmur of mitral stenosis or tricuspid stenosis. This murmur is low in frequency, and rumbling in quality. A continuous murmur usually begins during systole and extends through the second sound and throughout the diastolic period. It is usually produced as a result of one of four mechanisms: 1) An abnormal communication between an artery and vein; 2) An abnormal communication between the aorta and the right side of the heart or with the left atrium; 3) An abnormal increase in flow, or constriction in an artery; and 4) Increased or turbulent blood flow through veins. Patent Ductus Arteriosus (PDA) is the classical example of this murmur. This condition is usually corrected in childhood. It is heard best at base left, and is usually easily audible with the bell or diaphragm. Another example of a continuous murmur is the so-called venous hum, but in this instance one hears a constant roaring sound which changes little with the cardiac cycle. A late systolic murmur begins in the latter half of systole, peaks in the later third of systole, and extends to the second sound. It is a modified regurgitant murmur with a backward flow through an incompetent valve, usually the mitral valve. It is commonly heard in mitral valve prolapse, and is usually high in frequency (blowing in quality), and heard best with a diaphragm at the apex. It may radiate to the axilla or left sternal border. A pansystolic or holosystolic murmur is heard continuously throughout systole. It begins with the first heart sound, and ends with the second heart sound. It is commonly heard in mitral regurgitation, tricuspid regurgitation, and ventricular septal defect. This type of murmur is caused by backward blood flow. Since the pressure remains higher throughout systole in the ejecting chamber than in the receiving chamber, the murmur is continuous throughout systole. Diastolic murmurs are sustained noises that are audible between S2 and the next S. Unlike systolic murmurs, diastolic murmurs should usually be considered pathological, and not normal. Typical abnormalities causing diastolic murmurs are aortic regurgitation, pulmonic regurgitation, mitral stenosis, and tricuspid stenosis. The timing of diastolic murmurs is the primary concern of this program. These murmurs can be early, mid, late and pan in nature. In a pericardial friction rub, there are three sounds, one systolic, and two diastolic. The systolic sound may occur anywhere in systole, and the two diastolic sounds occur at the times the ventricles are stretched. This stretching occurs in early diastole, and at the end of diastole. The pericardial friction rub has a scratching, grating, or squeaking leathery quality. It tends to be high in frequency and best heard with a diaphragm. A pericardial friction rub is a sign of pericardial inflammation and may be heard in infective pericarditis, in myocardial infarction, following cardiac surgery, trauma, and in autoimmune problems such as rheumatic fever.

In addition to heart sound analysis, the timing between the onset and offset of particular features of the ECG (referred to as an interval) provides a measure of the state of the heart and can indicate the presence of certain cardiological conditions. An EKG analyzer is provided to interpret EKG/ECG data and generate warnings if needed. The analyzer examines intervals in the ECG waveform such as the QT interval and the PR interval. The QT interval is defined as the time from the start of the QRS complex to the end of the T wave and corresponds to the total duration of electrical activity (both depolarization and repolarization) in the ventricles. Similarly, the PR interval is defined as the time from the start of the P wave to the start of the QRS complex and corresponds to the time from the onset of atrial depolarization to the onset of ventricular depolarization. In one embodiment, hidden Markov and hidden semi-Markov models are used for automatically segmenting an electrocardiogram waveform into its constituent waveform features. An undecimated wavelet transform is used to generate an overcomplete representation of the signal that is more appropriate for subsequent modelling. By examining the ECG signal in detail it is possible to derive a number of informative measurements from the characteristic ECG waveform. These can then be used to assess the medical well-being of the animal or wearer. The wavelet methods such as the undecimated wavelet transform, can be used instead of raw time series data to generate an encoding of the ECG which is tuned to the unique spectral characteristics of the ECG waveform features. The segmentation process can use of explicit state duration modelling with hidden semi-Markov models. Using a labelled data set of ECG waveforms, a hidden Markov model is trained in a supervised manner. The model was comprised of the following states: P wave, QRS complex, T wave, U wave, and Baseline. The parameters of the transition matrix aij were computed using the maximum likelihood estimates. The ECG data is encoded with wavelets from the Daubechies, Symlet, Coiflet or Biorthogonal wavelet families, among others. In the frequency domain, a wavelet at a given scale is associated with a bandpass filter of a particular centre frequency. Thus the optimal wavelet basis will correspond to the set of bandpass filters that are tuned to the unique spectral characteristics of the ECG. In another implementation, a hidden semi-Markov model (HSMM) is used. HSMM differs from a standard HMM in that each of the self-transition coefficients aii are set to zero, and an explicit probability density is specified for the duration of each state. In this way, the individual state duration densities govern the amount of time the model spends in a given state, and the transition matrix governs the probability of the next state once this time has elapsed. Thus the underlying stochastic process is now a “semi-Markov” process. To model the durations of the various waveform features of the ECG, a Gamma density is used since this is a positive distribution which is able to capture the skewness of the ECG state durations. For each state i, maximum likelihood estimates of the shape and scale parameters were computed directly from the set of labelled ECG signals.

In addition to providing beat-to-beat timing information for other sensors to use, the patterns of the constituent waveform features determined by the HMM or neural networks, among other classifiers, can be used for detecting heart attacks or stroke attacks, among others. For example, the detection and classification of ventricular complexes from the ECG data is can be used for rhythm and various types of arrhythmia to be recognized. The system analyzes pattern recognition parameters for classification of normal QRS complexes and premature ventricular contractions (PVC). Exemplary parameters include the width of the QRS complex, vectorcardiogram parameters, amplitudes of positive and negative peaks, area of positive and negative waves, various time-interval durations, amplitude and angle of the QRS vector, among others. The EKG analyzer can analyze EKG/ECG patterns for Hypertrophy, Enlargement of the Heart, Atrial Enlargement, Ventricular Hypertrophy, Arrhythmias, Ectopic Supraventricular Arrhythmias, Ventricular Tachycardia (VT), Paroxysmal Supraventricular Tachycardia (PSVT), Conduction Blocks, AV Block, Bundle Branch Block, Hemiblocks, Bifascicular Block, Preexcitation Syndromes, Wolff-Parkinson-White Syndrome, Lown-Ganong-Levine Syndrome, Myocardial Ischemia, Infarction, Non-Q Wave Myocardial Infarction, Angina, Electrolyte Disturbances, Heart Attack, Stroke Attack, Hypothermia, Pulmonary Disorder, Central Nervous System Disease, or Athlete's Heart, for example.

In one implementation, an HMM is used to track animal motor skills or movement patterns. Animal movement involves a periodic motion of the legs. Regular walking involves the coordination of motion at the hip, knee and ankle, which consist of complex joints. The muscular groups attached at various locations along the skeletal structure often have multiple functions. The majority of energy expended during walking is for vertical motion of the body. When a body is in contact with the ground, the downward force due to gravity is reflected back to the body as a reaction to the force. When a person stands still, this ground reaction force is equal to the person's weight multiplied by gravitational acceleration. Forces can act in other directions. For example, when we walk, we also produce friction forces on the ground. When the foot hits the ground at a heel strike, the friction between the heel and the ground causes a friction force in the horizontal plane to act backwards against the foot. This force therefore causes a breaking action on the body and slows it down. Not only do people accelerate and brake while walking, they also climb and dive. Since reaction force is mass times acceleration, any such acceleration of the body will be reflected in a reaction when at least one foot is on the ground. An upwards acceleration will be reflected in an increase in the vertical load recorded, while a downwards acceleration will reduce the effective body weight. Zigbee wireless sensors with tri-axial accelerometers are mounted to the animal or wearer on different body locations for recording, for example the tree structure as shown in FIG. 16D. As shown therein, sensors can be placed on the four branches of the links connect to the root node (torso) with the connected joint, left shoulder (LS), right shoulder (RS), left hip (LH), and right hip (RH). Furthermore, the left elbow (LE), right elbow (RE), left knee (LK), and right knee (RK) connect the upper and the lower extremities. The wireless monitoring devices can also be placed on upper back body near the neck, mid back near the waist, and at the front of the right leg near the ankle, among others.

The sequence of animal motions can be classified into several groups of similar postures and represented by mathematical models called model-states. A model-state contains the extracted features of body signatures and other associated characteristics of body signatures. Moreover, a posture graph is used to depict the inter-relationships among all the model-states, defined as PG(ND,LK), where ND is a finite set of nodes and LK is a set of directional connections between every two nodes. The directional connection links are called posture links. Each node represents one model-state, and each link indicates a transition between two model-states. In the posture graph, each node may have posture links pointing to itself or the other nodes.

In the pre-processing phase, the system obtains the animal body profile and the body signatures to produce feature vectors. In the model construction phase, the system generate a posture graph, examine features from body signatures to construct the model parameters of HMM, and analyze animal body contours to generate the model parameters of ASMs. In the motion analysis phase, the system uses features extracted from the body signature sequence and then applies the pre-trained HMM to find the posture transition path, which can be used to recognize the motion type. Then, a motion characteristic curve generation procedure computes the motion parameters and produces the motion characteristic curves. These motion parameters and curves are stored over time, and if differences for the motion parameters and curves over time is detected, the system then runs the animal or wearer through additional tests to confirm athletic performance failure before recommending a doctor visit.

FIG. 2B shows a learning system for recommending treatment based on sensor data captured over time and based on treatment data for a population of animals. In FIG. 2B, during examination, a doctor uses a smartphone to review sensor data from the animal. Feature extraction is done on the data as detailed herein. In parallel, clinical information such as sex, age, temperature, medical history, among others, are provided to feature extraction. As the data is text, the feature extraction can be done by extracting feature windows around a particular word of interest. The description can be vectorized into a sparse two-dimensional matrix suitable for feeding into a classifier. Feature hashing, where instead of building a hash table of the features encountered in training, as the vectorizers do, instances of FeatureHasher apply a hash function to the features to determine their column index in sample matrices directly. Since the hash function might cause collisions between (unrelated) features, a signed hash function is used and the sign of the hash value determines the sign of the value stored in the output matrix for a feature. This way, collisions are likely to cancel out rather than accumulate error, and the expected mean of any output feature's value is zero.

In addition, prior examination data can be featurized. At the time of a given exam, relevant information for predicting the diagnosis or prognosis may come not only from the current exam, but also from the results of past exams. The system combines information from the current and past exams when making a prediction of diagnosis or prognosis. If all vetenary patients received regular exams, for example, annually, it would be possible to simply generate one feature vector for the current exam, another for the exam from 1 year ago, another for the exam from 2 years ago, etc. Those feature vectors could then be combined via simple concatenation (possibly followed by dimensionality reduction) using the same procedure described herein to combine features within a single exam to form a combined feature vector. However, in general, patients may not be expected to all have had regular past exams on the same schedule. For example, patient A may have had annual exams, patient B may have had exams every other year, and patient C may have only had exams during periods of illness, which occurred at irregular intervals. Therefore, there is a need for a consistent method of converting information from past exams into a feature vector in a way that does not depend on the frequency or interval between past exams. One possible method for combining information from past exams is to combine features from past exams via a weighted average that takes into account the time from the current exam, with more recent exams weighted higher. For example, a linear weighting function could be used which linearly runs from 0 at birth to 1 at the present time. For an example patient of age 10 who had exams at ages 3 months, 9 months, and 6 years, each feature would be averaged together across exams (excluding the present exam), with weights of 0.025, 0.075 and 0.6. Weighting functions other than linear could be used (e.g., logarithmic, power law, etc.) and weights could also be normalized to add up to 1. Features from the current exam would also be included separately in the feature vector, concatenated together with the weighted features from past exams. Alternatively, one could include the current exam's features in the weighted feature vector from past exams, instead of including it separately. The generated feature vectors are then provided to a deep learning system.

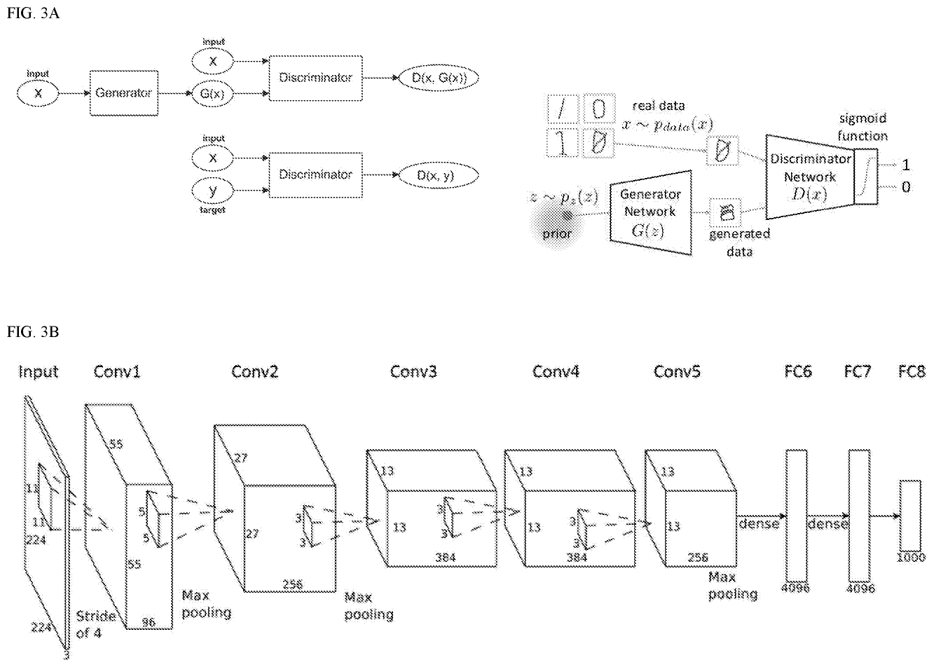

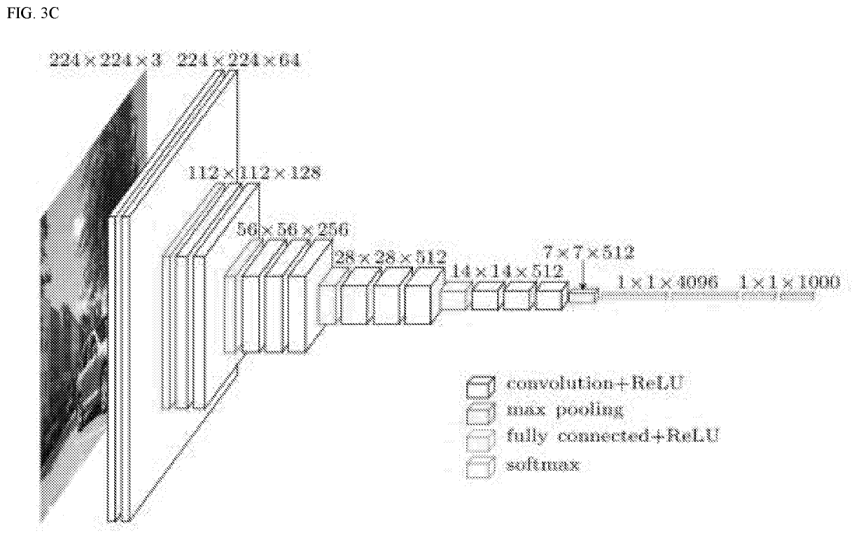

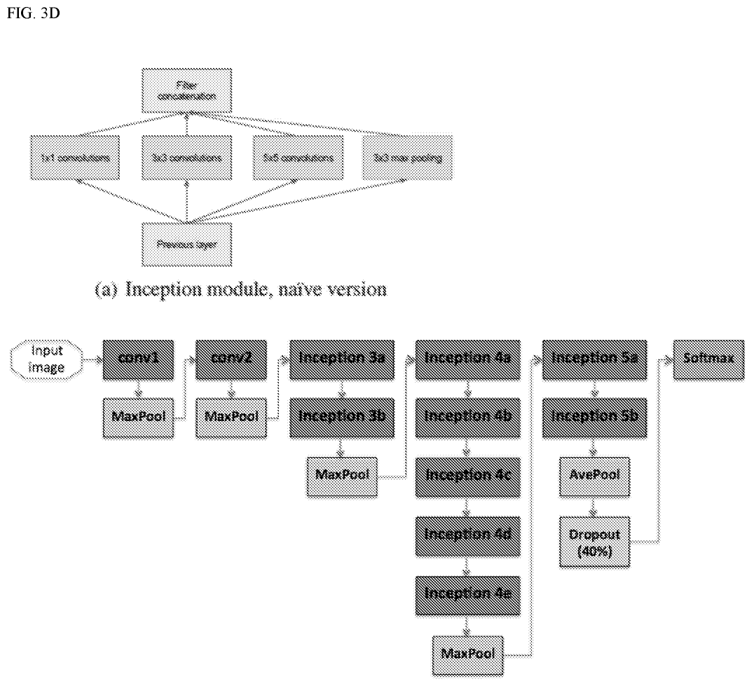

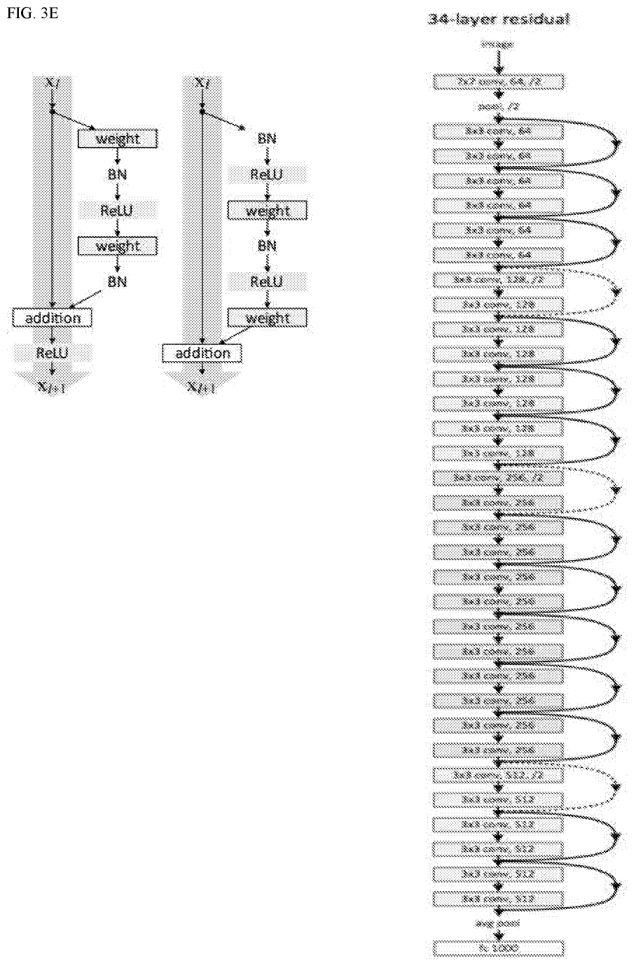

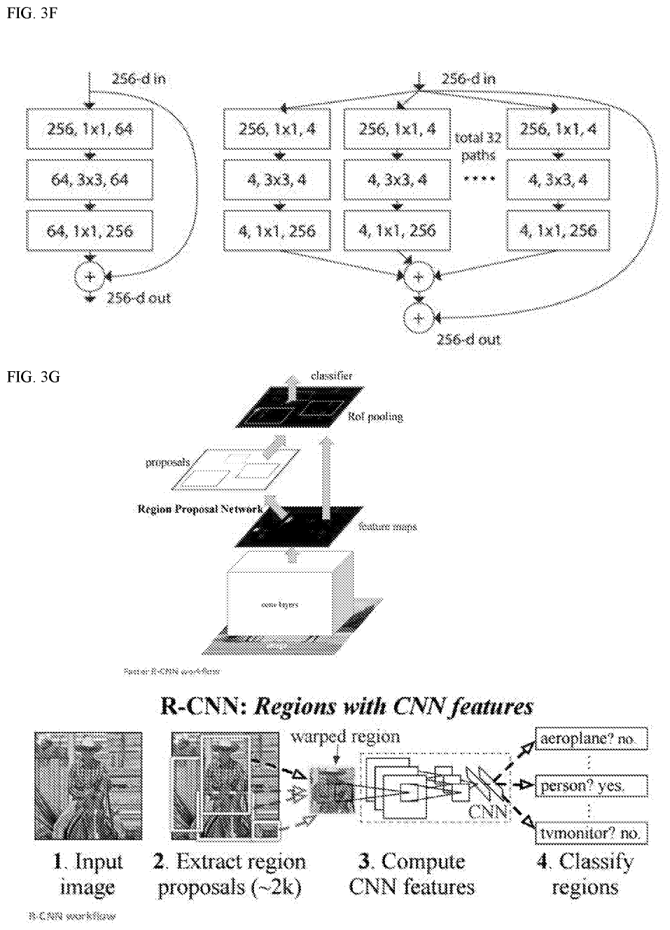

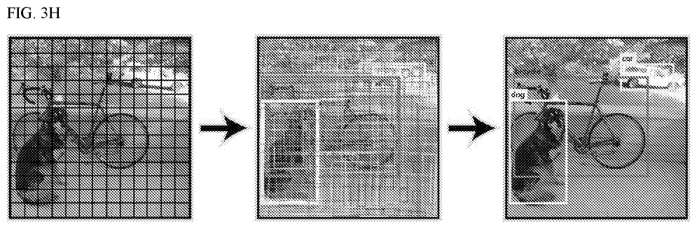

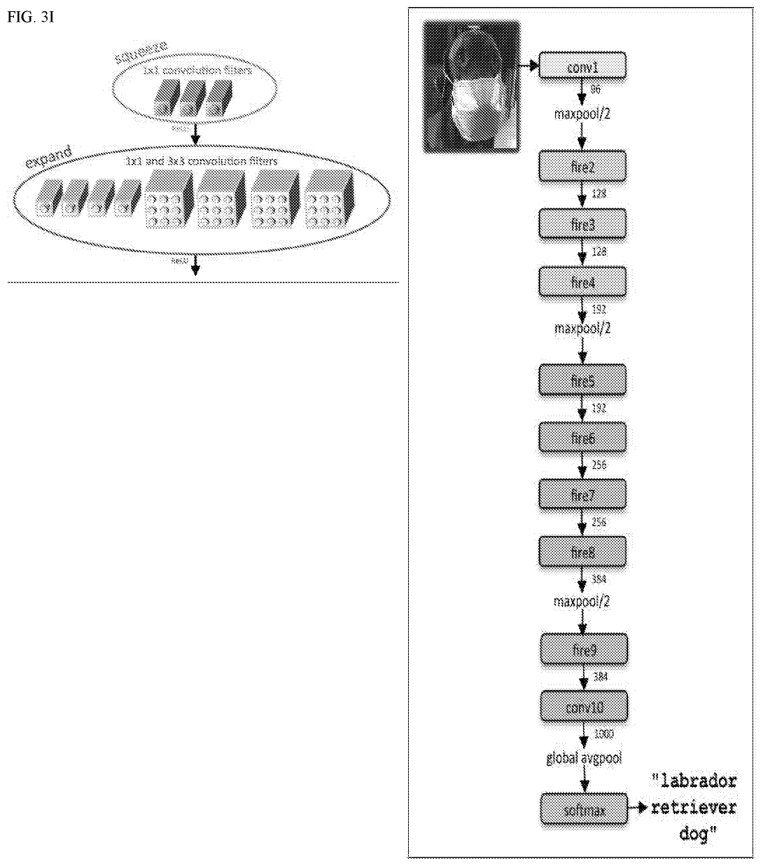

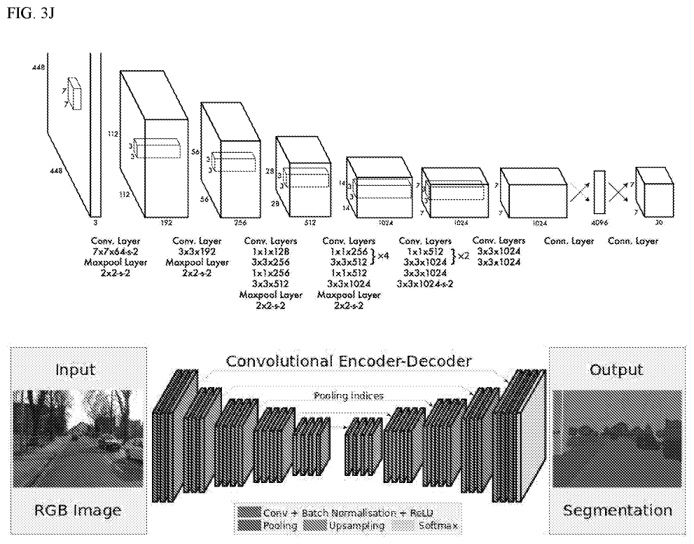

One embodiment uses a conditional-GAN (cGAN) as a deep learning machine. As shown in FIG. 3A, the cGAN consists of two major parts: generator G and discriminator D. The task of generator is to produce an image indistinguishable from a real image and “fool” the discriminator. The task of the discriminator is to distinguish between real image and fake image from the generator, given the reference input image.

The objective of a conditional-GAN is composed of two parts: adversarial loss and LI loss. The adversarial loss can be: [symbol] _{cGAN}(G,D)=E_{x,y}[log D(x,y)]+Eₓ[log(1−D(x,G(X))] where L1 distance is added to generated image. L1 distance is preferred over L2 distance as it produces images with less blurring. Thus our full objective for the minimax game is:

$$( {G}^{*} , {D}^{*} ) = arg {min}_{G} {max}_{D} ( {ℒ}_{cGAN} ( G , D ) + λ {ℒ}_{L 1} ( G ) )$$

The ResNet-50 network by He et al. can be used as the generator, while the discriminator can be a convolutional “PatchGAN” classifier with architecture similar to the classifier in pix2pix as our discriminator.

In addition to cGAN, other neural networks can be used. FIGS. 3B-3J show exemplary alternatives, including:

1. AlexNet—AlexNet is the first deep architecture which can be introduced by one of the pioneers in deep learning—Geoffrey Hinton and his colleagues. It is a simple yet powerful network architecture, which helped pave the way for groundbreaking research in Deep Learning as it is now.

2. VGG Net—The VGG Network can be introduced by the researchers at Visual Graphics Group at Oxford (hence the name VGG). This network is specially characterized by its pyramidal shape, where the bottom layers which are closer to the image are wide, whereas the top layers are deep. VGG contains subsequent convolutional layers followed by pooling layers. The pooling layers are responsible for making the layers narrower. In their paper, they proposed multiple such types of networks, with change in deepness of the architecture.

3. GoogleNet—In this architecture, along with going deeper (it contains 22 layers in comparison to VGG which had 19 layers), the Inception module is used. In a single layer, multiple types of “feature extractors” are present. This indirectly helps the network perform better, as the network at training itself has many options to choose from when solving the task. It can either choose to convolve the input, or to pool it directly. The final architecture contains multiple of these inception modules stacked one over the other. Even the training is slightly different in GoogleNet, as most of the topmost layers have their own output layer. This nuance helps the model converge faster, as there is a joint training as well as parallel training for the layers itself.

4. ResNet—ResNet is one of the monster architectures which truly define how deep a deep learning architecture can be. Residual Networks (ResNet in short) consists of multiple subsequent residual modules, which are the basic building block of ResNet architecture. ResNet uses of standard SGD instead of a fancy adaptive learning technique. This is done along with a reasonable initialization function which keeps the training intact; Changes in preprocessing the input, where the input is first divided into patches and then feeded into the network. The main advantage of ResNet is that hundreds, even thousands of these residual layers can be used to create a network and then trained. This is a bit different from usual sequential networks, where you see that there is reduced performance upgrades as you increase the number of layers.

5. ResNeXt—ResNeXt is said to be the current state-of-the-art technique for object recognition. It builds upon the concepts of inception and resnet to bring about a new and improved architecture.

6. RCNN (Region Based CNN)—Region Based CNN architecture is said to be the most influential of all the deep learning architectures that have been applied to object detection problem. To solve detection problem, what RCNN does is to attempt to draw a bounding box over all the objects present in the image, and then recognize what object is in the image.

7. YOLO (You Only Look Once)—YOLO is a real time system built on deep learning for solving image detection problems. As seen in the below given image, it first divides the image into defined bounding boxes, and then runs a recognition algorithm in parallel for all of these boxes to identify which object class do they belong to. After identifying this classes, it goes on to merging these boxes intelligently to form an optimal bounding box around the objects. All of this is done in parallely, so it can run in real time; processing up to 40 images in a second.

8. SqueezeNet—The squeezeNet architecture is one more powerful architecture which is extremely useful in low bandwidth scenarios like mobile platforms. This architecture has occupies only 4.9 MB of space, on the other hand, inception occupies ˜100 MB! This drastic change is brought up by a specialized structure called the fire module which is good for mobile phone.

9. SegNet—SegNet is a deep learning architecture applied to solve image segmentation problem. It consists of sequence of processing layers (encoders) followed by a corresponding set of decoders for a pixelwise classification. Below image summarizes the working of SegNet. One key feature of SegNet is that it retains high frequency details in segmented image as the pooling indices of encoder network is connected to pooling indices of decoder networks. In short, the information transfer is direct instead of convolving them. SegNet is one the best model to use when dealing with image segmentation problems.

With accelerometers and gyroscopes, the system can analyze animal locomotion, which requires the measurement and analysis of the following: Temporal characteristics, Electromyographic signals, Kinematics of limb segments, and Kinetics of the foot-floor and joint resultants. Temporal analysis of gait in the dog has yielded some norms for the average velocity of walking as well as time durations for the two phases of gait: the stance phase and the swing phase. The symmetry and asymmetry of gait can be captured by the accelerometer.

The system can model the animal's kinematics or relative motion that exists between rigid bodies, known as links between the body and the legs. Kinematic analysis of gait involves accelerometers positioned at different animal body parts to capture the displacement, velocity, and acceleration of various body segments. A model of the links and their movements can be created for diagnosis and also for real time assistance if needed. The model can also be used for energy consumption analysis, athletic training, and predictive health for particular tasks.

In one embodiment, the gaits of the dog are commonly used patterns of locomotion that can be divided into two main groups: symmetric and asymmetric. With symmetric gaits such as the walk, trot, and pace, the movement of the limbs on one side of the dog's body repeats the motion of the limbs on the opposite side with the intervals between foot falls being nearly evenly spaced. With asymmetric gaits such as the gallop, the limb movements of one side do not repeat those of the other and the intervals between foot falls are unevenly spaced. When considering gaits, one full cycle is referred to as a stride.

Most dogs stand squarely over their forelegs and hindlegs at rest; this is also true during walking, since the dog will support his body by three or more legs. However, as the animal increases its speed and changes gait, it has less support; therefore the legs move toward the center of mass, which is directly below the body. The gait pattern, called single tracking, is used to decrease the lateral oscillations of the body and provide continual support of the center of mass. The degree of convergence of the limbs toward the center line under the middle of the body depends on both the speed of the animal and the conformation. The walk has been described as the least tiring and most efficient form of locomotion of the dog. The trot is a symmetric gait produced when the diagonal pairs of legs move almost simultaneously, causing the duration of contact with the ground to be slightly longer for the hindlegs than the forelegs. The pace is a symmetric gait in which support is maintained by the animal with lateral pairs of legs and the animal moves by swinging the forelimb and hindlimb on one side while bearing weight on the other side. It is a gait commonly used in long-legged dogs with close-coupled bodies and allows the animal to move in a straight, forward direction without the interference between front and hind legs that may occur at a trot. The gallop is an asymmetric gait used for high-speed locomotion. There are two patterns of gallop in the dog: the transverse gallop similar to the pattern used by the horse; and the rotary gallop which seems to be preferred by the dog and which in the horse is referred to as a crossed-lead gallop. The dog can sustain the gallop at two speeds. The slow gallop, known as a canter or lope, represents a gait that can be sustained easily over a long period of time. It is a submaximal form of aerobic exercise in which aerobic glycolysis contributes to the total power of the dog while running. The fastest gallop can be maintained for short periods owing to the contribution of anaerobic glycolysis during exercise intensities that are greater than maximal aerobic exercise can sustain.

The system can detect neurologic problems, as almost every neurologic condition will be associated in some way with an abnormality of gait, such as an inability to gait, knuckling, lameness, unsteadiness, or development of a protective mode of walking evidencing severe pain. Arching of the back, lowering of the head and neck, and extension of the head are seen with intervertebral disk disease, especially cervical disease but also with thoracolumbar disease. The early signs of degenerative myelopathy are usually gait abnormalities of the hindleg. These abnormalities are especially evident when the dog is trotting or moving in a circular direction. The hindlimbs seem unstable, and the legs seem to lose their proprioceptive ability. Knuckling of the hindlimbs is also characteristic of the problem, and the hindfeet should be observed for evidence of scraping of the nails. In some dogs, the sound of toenails dragging on a hard floor is quite noticeable and should alert the clinician to the fact that a neurologic problem may exist, since lame dogs rarely knuckle or drag their feet when walking unless neurologic disease is present.

While the above embodiments are described for an animal, suitably modifications can be done for human implant as well. Such modifications include use of biomaterials to prevent implant rejection (such as those in breast implants, for example). A slow release medication can be provided to avoid tissue rejection. Also, the HR and RR can be modified for human patterns.

In some embodiments, the system may include a substrate such as a circuit board (e.g., a printed circuit board (PCB) or flexible PCB) on which circuit components (e.g., analog and/or digital circuit components) may be mounted or otherwise attached. However, in some alternative embodiments, the substrate may be a semiconductor substrate having circuitry fabricated therein. The circuitry may include analog and/or digital circuitry. Also, in some semiconductor substrate embodiments, in addition to the circuitry fabricated in the semiconductor substrate, circuitry may be mounted or otherwise attached to the semiconductor substrate. In other words, in some semiconductor substrate embodiments, a portion or all of the circuitry, which may include discrete circuit elements, an integrated circuit (e.g., an application specific integrated circuit (ASIC)) and/or other electronic components (e.g., a non-volatile memory), may be fabricated in the semiconductor substrate with the remainder of the circuitry being secured to the semiconductor substrate 116 and/or a core (e.g., ferrite core) for the inductive element. In some embodiments, the semiconductor substrate and/or a core may provide communication paths between the various secured components.

While the system described above is susceptible to various modifications and alternative constructions, certain illustrated embodiments thereof are shown in the drawings and have been described above in detail. It should be understood, however, that there is no intention to limit the system to the specific form or forms disclosed, but on the contrary, the intention is to cover all modifications, alternative constructions, and equivalents falling within the spirit and scope of the above system.

What is claimed is:

1. A system to monitor an animal or a biological subject, comprising:
an implantable device to be inserted inside the subject, the device including an implanted transceiver, an accelerometer, one or more sensors, a battery to power the transceiver, accelerometer and one or more sensors, and a wireless charger to charge the battery; and
a wireless charging system outside of the subject to charge the battery in the implantable device.

2. The system of claim 1, wherein the device is implanted proximal to a shoulder blade or a dorsal midline of the subject.

3. The system of claim 1, wherein the device is implanted proximal to a neck area, a shoulder blade area, or an area of the subject not accessible to the animal through chewing or biting.

4. The system of claim 1, comprising a temperature sensor, heart rate sensor, a hydration sensor, impedance sensor, EKG sensor, or a pulse oximetry sensor.

5. The system of claim 1, comprising a temperature sensor, heart rate sensor, and a pulse oximetry sensor.

6. The system of claim 1, comprising a blood pressure sensor or a glucose sensor.

7. The system of claim 1, comprising a pulse oximetry sensor coupled to a processor to determine breathing rate from the pulse oximetry sensor.

8. The system of claim 1, wherein the implanted transceiver comprises a personal area network or a wireless local area network.

9. The system of claim 1, wherein the wireless charger comprises an inductive charger or a capacitive charger.

10. The system of claim 1, comprising a pacemaker circuit.

11. The system of claim 1, comprising a neck strap or a vest having an area within charging range of the wireless charger.

12. The system of claim 11, wherein the strap or vest comprises a temperature sensor, a chest mounted accelerometer to detect breathing, and an EKG sensor.

13. The system of claim 1, wherein the wireless charging system is carried by the strap or vest.

14. The system of claim 1, comprising a cellular transceiver or a satellite network transceiver in the strap or vest to provide data transmission.

15. The system of claim 1, comprising a glucose sensor communicating data to a remote device to coordinate physical activity or exercise proximal to a meal to adjust glucose level without medication.

16. The system of claim 1, comprising a Generative Adversarial Networks (GANs), a recurrent neural network, a statistical recognizer, a learning machine, or a neural network to determine health issue from the sensor.

17. The system of claim 1, comprising one or more medical reservoirs and one or more pumps to dispense medication.

18. The system of claim 18, wherein the medication comprises insulin, blood pressure medication, stroke medication, coronary artery medication, cancer medication, respiratory medication, obstructive pulmonary medication, and Alzheimer medication.

19. The system of claim 1, comprising a glucose sensor coupled to an insulin reservoir to dispense insulin in a closed loop.

20. The system of claim 19, comprising a pacemaker coupled to the glucose sensor, wherein pacemaker operation is adjusted based on glucose level.
